(19) United States
(12) Patent Application Publication
(10) Pub. No.: US 2006/0156438 A1
(43) Pub. Date: Jul. 13, 2006

(54) Protein containing a burp domain

(76) Inventors: Gopalan Selvaraj, Saskatoon (CA); Aiming Wang, London (CA); Gun Xia, Saskatoon (CA); Wenshuang Xie, Richmond, CA (US); Raju Datla, Saskatoon (CA)

Correspondence Address: BORDEN LADNER GERVAIS LLP, 1100-100 QUEEN ST, OTTAWA, ON, K1P 1J9, CA

(21) Appl. No.: 10/522,894

(22) PCT Filed: Aug. 1, 2003
(86) PCT No.: PCT/CA03/01169

§ 371 Date: Oct. 3, 2005

Related U.S. Application Data
(60) Provisional application No. 60/400,836, filed on Aug. 2, 2002.

(51) Int. Cl. A01H 1/00; C12N 5/04; C07K 14/415

(52) U.S. Cl. 800/287; 435/468; 435/419; 530/370

(57) ABSTRACT

The anther recruits a number of specific-genes to support microspore development. Here we report identification of a novel gene, RAFTIN1, from bread wheat and rice, encoding a protein containing a BURP domain hallmarked with 4 CII repeats at its C-terminus. This single copy gene (per haploid complement in cereals) is exclusively expressed in the tapetum during postmeiotic stages when the young microspore undergoes rapid expansion. RAFTIN1 is biosynthesized in the tapetum, transported into the Ubisch body, and further deposited onto the microspore exinewall. Transgenic rice, in which RAFTIN1 expression is down-regulated, shows normal growth and development hut also shows male sterility. In the RAFTIN1-less anther, tapetal degeneration is retarded and pollen lacks contents. Thus, RAFTIN1 assembly in the Ubisch body and the microspore exinewall is required for microspore development, probably for regulation of metabolite transport from the tapetum to the microspore. Silencing or knocking-out of RAFTIN will find utility in breeding programs wherever male sterile lines are required.

### TECHNICAL FIELD

This invention generally relates to a method of modulating male fertility in plants, and more specifically relates to a method of modulating male fertility in cereal plants by down regulating expression of RAFTIN genes. Modulation of male fertility in plants is of increasing significance and utility in controlled plant breeding programs in which male sterile lines are useful. Male sterile lines are of interest and use commercially and can be useful in reducing unwanted gene flow among different breeds of the same species of plant, for example from a transgenic crop to its non-transgenic neighbours.

### BACKGROUND

The anther, a tiny, short-lived but functionally complex organ encompassing the male gametophyte plays a fundamental role in the reproductive cycle of flowering plants as well as in the agricultural practice, i.e. hybrid seed production. As one of the most elaborate and complex processes in plant life, anther development, in particular at the molecular level still remains poorly understood. Previously cytological observations through the light and electron microscopes have sketched a generalized scheme of developmental events leading to the ontogeny of the anther architecture for most angiosperms (D'Arcy, 1996, Goldberg et al., 1993, Shivanna et al., 1997). Differentiating from stamen primordia, the anther initially consists of a mass of homogeneous cells surrounded by the epidermis. Followed with the differentiation of archesporial cells in the hypodermal region, a four-lobed structure (four microsporangia or locules) is developed. A periclinal division of archesporial cells gives rise to the formation of the outer, primary parietal layer and the inner, sporogenous layer. The former, upon cell divisions generates multi-layer cells which are differentiated into the endothecium (the outermost layer beneath the epidermis), the middle layer (one to three layers between the endothelium and the tapetum) and the tapetum (the innermost layer). Concomitant with the differentiation of the anther wall (the epidermis, the endothecium and the middle layer) and the tapetum, the centered sporogenous layer gives rise to microspore mother cells (MMCs). The differentiated MMC undergoes meiosis to produce tetrads of microspores. Further development from young microspores to mature pollen grains, morphologically evident with the enlargement of the anther and microspores, the degeneration of the tapetum, pollen desiccation, anther dehiscence and pollen release, is an exquisite interplay between the male gametophyte and sporophytic tissues of the anther (Goldberg et al., 1993, McCormick, 1993).

A rapidly increasing body of evidence suggests that the development of normal pollen grains requires a functional tapetum (Aarts et al., 1997, De Block et al., 1997, Goldberg et al., 1993, Mariani et al., 1992, Mizelle et al., 1989). As early as prior to the onset of meiosis, the tapetum tissue initially networks meitocytes via plasmodesmata with the rest of the supporting tissues of the anther. Upon the formation of postmeiotic tetrads, the tapetum functions as nursing cells to produce callase to disassociate the tetrads. During the following free microspore and vacuolated microspore stages, the tapetum supplies essential nutrients and metabolites to assist in microspore expansion, vacuolation and extracellular matrix formation (Mascarenhas, 1990, Mizelle et al., 1989, Sanders et al., 1999). Towards the end of the vacuolated microspore stage, the tapetum triggers programmed degeneration that continues throughout the vacuolated pollen grain stage, releasing its macromolecules such as proteins, lipids and carbohydrates, some of them utilized in the build-up of the complex extracellular wall surrounding the microspore (Furness & Rudall, 2001, Mascarenhas, 1975, McCormick, 1993, Mizelle et al., 1989, Piffanelli et al., 1997, Shivanna et al., 1997, Wiermann & Gubatz, 1992). However, by what means the tapetal metabolites are transported onto the surface of microspore is obscure and how the sporophytically produced, gametophytically localized proteins contribute to microspore development is not known.

In this study, we report the isolation of three apparently homologous anther-specific genes taRAFTIN1a, taRAFTIN1b and taRAFTIN1d in allohexaploid wheat (Triticum aestivum L.) and their ortholog osRAFTIN1 in rice (Oryza sativa L.). We provide evidence that RAFTIN1 transcription only took place in the tapetum but their proteins were predominantly evident in the tapetum, the Ubisch body and the microspore exinewall, establishing an example of the tapetum-Ubisch Body-microspore transport pathway. Our data show that RAFTIN1, whose structural counterpart is not found in the Arabidopsis genome or other eudicot genomes but found in the ESTs derived from the anther or anther-containing reproductive tissues of various monocot species including barley, sorghum, hexaploid wheat, rice, wild diploid wheats, maize and rye contains a BURP domain that has been only found in the plant kingdom, thus constituting the first anther-specific version of BURP domain proteins. Our results demonstrate that in the transgenic rice where RAFTIN1 expression was down-regulated, the normal tapetal degeneration was retarded and microspore contents were lacking, leading to the production of male sterile pollen. We suggest that the Ubisch body- and microspore exinewall-assembled RAFTIN1 proteins are probably involved in the transport of the metabolites from the tapetum to microspore, that is required for anther development in rice, wheat and probably in other cereal species. This will prove useful in manipulating male sterility in such other plants such as species of monocotyledon, e.g. rye, oats, barley, sorghum and maize (in addition to wheat and rice).

### BRIEF DESCRIPTION OF THE DRAWINGS

FIG. 1. Genomic Organization of RAFTIN1 Gene Family in Wheat and Rice. (A) Schematic representation of genomic structures of cloned RAFTIN1 genes in wheat and rice. taRAFTIN1a and taRAFTIN1b were isolated from a hexaploid wheat, and osRAFTIN1 from rice. The numbers denote nt sequence length of the untranslated regions (UTR), exons (boxes) and introns (triangles). Not drawn to scale. (3) RAFTIN 1 gene in rice. Southern blot of rice genomic DNA (˜10 μg) digested with HindIII (H), EcoRI (E) and BamHI (B) and probed with the entire osRAFTIN1 ORF retrieved by PCR; size markers are from a λ DNA-HindIII digest. (C) taRAFTIN1a and closely related sequences in hexaploid, tetraploid and diploid wheats, and diploid rice. Southern blot of genomic DNA (˜15 μg for wheat and ˜10 μg for rice) restricted with HindIII (H) and EcoRI (E) and probed with the entire taRAFTIN1a ORF retrieved by PCR; size markers are from a λ DNA-HindIII digest.

FIG. 2. Anther-Specific Expression of RAFTIN1 in Wheat and Rice. (A) and (C) Northern blot analysis of RAFTIN1 expression in hexaploid wheat and rice. Total RNA (5 μg) from each tissue was loaded. (A) probed with the entire taRAFTIN1a ORF retrieved by PCR. (C) probed with the entire osRAFTIN1 ORF retrieved by PCR. (B) and (D) RT-PCR analysis of RAFTIN1 expression in wheat and rice. (B) in wheat. (D) in rice. Primers and PCR conditions were described in METHODS. Total RNA isolated from different tissues indicated here was used for RT-PCR and northern blotting analyses. Root, root tissue; Stem, stem tissue; Leaf, leaf tissue; Flower, developing young flower tissue; Fl w/o anther, developing young flower-tissue with anther removed; Anther, developing anther tissue. For northern blot analyses, the panel underneath is the same gel used for blotting stained with ethidium bromide. For RT-PCR, the panel underneath is the control RT-PCR of a housekeeping gene, GAPDH (glyceradehyde-3-phosphate dehydrogenase, taGAPDH for wheat, osGAPDH for rice).

FIG. 3. Detection of RAFTIN1 Proteins in Wheat Anther. Protein extraction and western blot analysis were performed as described in METHODS. Purified taRAFTIN1a antiserum IgG was used for detection. Total proteins were extracted from different wheat tissues as indicated in FIG. 3.

FIG. 4. Amino Acid Sequences of RAFTIN1 Proteins and BURP Domains. (A) Comparison of the deduced primary structure of RAFTIN1 gene products. The predicted transmembrane domains are indicated as a filled bar. The specific sequences of these regions in each gene product are boxed. (B) Alignment of BURP domains. Substitutions are shown as such: identity as hyphens, and the gaps introduced in the alignment as triangles. The highly conserved amino acids, and CH motifs are indicated as dots and stars, respectively.

FIG. 5. In situ RNA Hybridization of RAFTIN1 Transcripts and Immunocytochemical Detection of RAFTIN1 Proteins in Cross-Sections of Hexaploid Wheat Flowers. From (A) to (D), detection of RAFTIN1 mRNA in wheat young florescence. (A) Hybridized to a taRAFTIN1a antisense probe. (B) Hybridized to a taRAFTIN1a sense probe (control). (C) and (D) Enlargement of (A) and (B), respectively. (E) and (F) Detection of RAFTIN1 mRNA in rice young florescence. (E) Hybridized to an osRAFTIN1 antisense probe. (F) Hybridized to an osRAFTIN1 sense probe (control). From (G) to (M), detection of RAFTIN1 proteins in wheat young inflorescence by taRAFTIN1a polycolonal antibodies, except for (H) which has been probed with pre-immune sera (control). From (I) to (J), the sections showing various developmental stages, progressing from the left to the right, were chosen. (I), “tetrad” stage; (J), “free microspore” stage; (K), “vacuolated microspore” stage; (L), “vacuolated pollen grain” stage; (M), “3-nucleate pollen grain” stage. Purified taRAFTIN1a antiserum IgG was used for detection. The bluish precipitate due to positive reaction is indicated by solid arrows. tp: tapetum; ms, microspore; ps; pollen sac; pg: pollen grain; ep: epidermis; en: endothecium. In (A) and (B), scale bars=200 μm; in (C) and (D), =100 μm; all other panels: 40 μm.

FIG. 6. Electron Micrographs of Wheat Anther Tissues Labelled with Polyclonal Antibodies Raised against taRAFTIN1a. (A) Anther ultra-thin sections immunolabeled with anti-taRAFTIN1a IgG. x7531. (B) Tapetal cells and Ubisch bodies immunolabeled with anti-taRAFTIN1a IgG. x45500. (C) Exinewall of microspore immunolabeled with anti-taRAFTIN1a IgG. x29640. ep, epidermis; en, endothecium; ml, middle layer; ta, tapetum; ub, Ubisch body; ex, exine; ms, microspore; ob, orbicular wall; ba, bacule; te, tectum; and fl, foot layer.

FIG. 7. Silencing osRAFTIN1 in Transgenic Rice using an Intron-Containing Hairpin RNA Strategy. (A) Schematic representation of intron-containing hairpin constructs designed for genetic transformation. (B) Northern blot analysis of osRAFTIN1 expression in transgenic rice transformed with intron-containing hairpin constructs. Percentage of osRAFTIN1 expression in different lines relative to that of WT normalized against total RNA loaded is given.

FIG. 8. Phenotypes of Wild-Type Rice and osRAFTIN1-Silenced Lines. (A) Vegetative growth of a wild-type rice line (457-5) transformed with a hygromycin resistant gene. (B) Vegetative growth of a transgenic line (507-5) transformed with a 35S::hy-osRAFTIN1 chimeric gene. (C) Mature florescence of Line 457-5. (D) Mature florescence of Line 507-5. (E) Rice grains of Line 457-5. (F) Rice grains (empty) of Line 507-5. (G) Hulls and rice of (E). (H) Hulls and a few undeveloped embryos of (F). (I) Rice grains of Line 507-5 fertilized with wild-type pollen.

FIG. 9. Scanning Electron Micrographs of Anthers and Pollen Grains of Wild-Type Rice and osRAFTIN1-Silenced Lines. (A) A representative of mature anthers from wild-type rice. (B) A representative of pollen grains from a mature undehisced wild-type anther. (C) Surface of a mature pollen grain from a wild-type anther. (D) A representative of mature pollens from transgenic Line 507-5 in which osRAFTIN1 anther-specific expression was down-regulated by an intron-spliced hairpin RNA strategy. (E) A representative of pollen grains from a mature undehisced anther in Line 507-5. (F) Surface of a mature pollen grain from Line 507-5.

In (A) and (D), scale bars=1 mm; in (B) and (E), =10 μm; in (C) and (F), =1 μm.

FIG. 10. Transmission Electron Micrographs of Anthers and Pollen Grains of Wild-Type Rice and osRAFTIN1-Silenced Lines. (A) and (B) Wild-type. (C) and (D) osRAFTIN1-silenced line 507-5. Scale bars=10 μm. ep, epidermis; en, endothecium; ta, tapetum; ub, Ubisch body; ex, exine; ms, microspore; ob, orbicular wall.

FIG. 11. taRAFTIN1a cDNA sequence (1338 nt excluding the polyA tail, ORF from nt 29 to nt 1198) (SEQ ID NO: 29). Start codon and stop codon are underlined.

FIG. 12. taRAFTIN1a genomic sequence (1560 bps including two introns). Introns are shown in lower case letters (SEQ ID NO: 30). Start codon and stop codon are underlined.

FIG. 13. taRAFTIN1a promoter sequence (1719 bps) (SEQ ID NO: 31).

FIG. 14. taRAFTIN1b cDNA sequence (1275 bps excluding the polyA tail, ORF from nt 25 to nt 1113) (SEQ ID NO: 32). Start codon and stop codon are underlined.

FIG. 15. taRAFTIN1b genomic sequence (1503 bps including two introns). Introns are shown in lower case letters (SEQ ID NO: 33). Start codon and stop codon are underlined.

FIG. 16. taRAFTIN1b promoter sequence (2095 bps) (SEQ ID NO: 34).

FIG. 17. taRAFTIN1d predicted cDNA sequence (246 bps) (SEQ ID NO: 35).

FIG. 18. taRAFTIN1d partial genomic sequence (441 bps) (SEQ ID NO: 36). Introns are shown in lower case letters.

FIG. 19. osRAFTIN1 cDNA sequence (1301 bps, ORF from nt 63 to nt 1301) (SEQ ID NO: 37). Start codon and stop codon are underlined.

FIG. 20. osRAFTIN1 genomic sequence (1479 bps, two introns included) (SEQ ID NO: 38). Introns are shown in lower case letters.

FIG. 21. osRAFTIN1 promoter sequence (1461 bps) (SEQ ID NO: 39).

FIG. 22 represents predicted protein sequences of taRAFTIN1a (389 residues) (SEQ ID NO: 40), taRAFTIN1b (362 residues) (SEQ ID NO: 41), taRAFTIN1d (partial sequence, 82 residues) (SEQ ID NO: 42) and osRAFTIN1 (412 residues) (SEQ ID NO: 43).

### DETAILED DESCRIPTION OF THE INVENTION

Genomic Organization of RAFTIN1 in Hexaploid Wheat and Diploid Rice

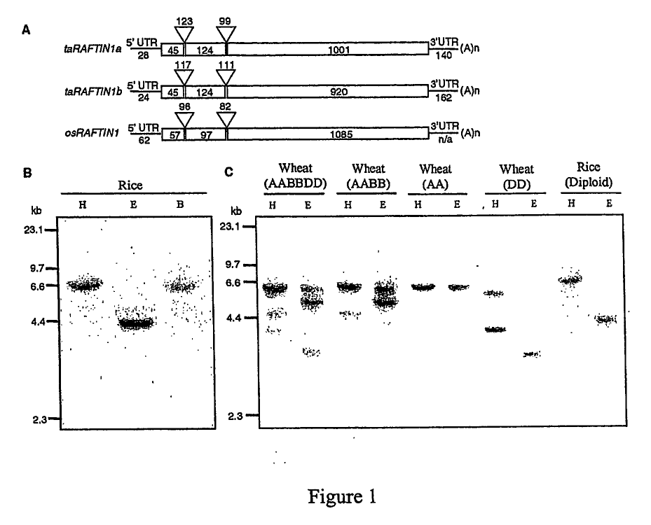

An anther-specific cDNA clone (A71) encoding a polypeptide was obtained from a wheat anther full-length cDNA library. Further 3 screens of the library using the 5′ region of the A71 as a probe identified, based on their sequence differences in 5′ and 3′ regions, two groups of cDNAs designated as taRAFTIN1a and taRAFTIN1b, respectively. The longest from each group, found to be full-length cDNA clones by the 5′ rapid amplification of cDNA ends (RACE) analysis, were completely sequenced. TaRAFTIN1a was 1356 nucleotides (nt) in length, and had a predicted open reading frame of 1170 nt (from nt 29 to 1198) encoding a protein with 389 amino acids (aa) (41.3 kDa) while taRAFTIN1b had a shorter cDNA (1285 nt) and a smaller ORF (1089 nt, from nt 25 to 1113) coding for 362 aa (38.3 kDa). Pairwise BLAST analysis of these two ORFs showed that they were 93% identical to each other at the nt level and 83% at the aa level. The corresponding genomic sequences of taRAFTIN1a and taRAFTIN1b cDNAs were retrieved from the wheat genome using a polymerase chain reaction (PCR) approach. Comparison of RAFTIN1 cDNA sequences with their corresponding genomic sequences revealed that both transcripts of taRAFTIN1a and taRAFTIN1b were interrupted by two introns in the 5′ regions of the ORFs and composed of 3 exons (FIG. 1A). During an endeavor to obtain more taRAFTIN1a and taRAFTIN1b related genes, a 701-bp genomic fragment (named taRAFTIN1d) whose predicted cDNA (246 bp) was 96% and 97% identical to the 5′ terminal coding regions of taRAFTIN1a and taRAFTIN1b, respectively, was isolated from the hexaploid wheat by PCR, whereas three additional screens of the anther cDNA library failed to isolate taRAFTIN1d cDNA or identify any new RAFTIN1-like genes. Since taRAFTIN1d had only the partial genomic sequence, it was not characterized further in this study.

As BLASTN searches of the public databases only identified a hypothetical gene (AP000364) obtained from the rice genome sequencing, hereafter named osRAFTIN1, the predicted coding region of osRAFTIN1 cDNA and its corresponding genomic DNA were cloned by RT-PCR and PCR, respectively. The ORF of osRAFTIN1, also interrupted by two introns at its 5′ terminal region is 1239 nt in length, encoding a 412 aa polypeptide with a predicted molecular mass of 42.3 kDa osRAFTIN ORF cDNA shared ˜66% identity to taRAFTIN1a and taRAFTIN1b ORFs. The polypeptide of osRAFTIN1 was overall ˜58% identical to taRAFTIN1a and taRAFTIN1b, and had a similar size and structure.

DNA gel blot analysis was performed to estimate the copy number of RAFTIN1 in rice and in the allohexaploid (AABBDD genome), tetraploid (AABB) and diploid wheats (AA or DD) using the coding region of osRAFTIN1 or taRAFTIN1 as a probe. The osRAFTIN1 probe hybridized to only one DNA fragment of the rice genome digested with each of 3 different restriction enzymes that do not cleave the coding region (FIG. 1B). All recognized bands were of the sizes consistent with those obtained through electronic southern analysis of the genomic sequences retrieved from the public domain (GenBank accession no: AP000364, Sasaki, T., Matsumoto, T. and Yamamoto, K., 1999). Thus, there only exists one copy of RAFTIN1 gene in the rice genome. When the same digestion was probed with the wheat taRAFTIN1a ORF cDNA, the probe specifically hybridized to the DNA fragments with the same sizes as recognized by the osRAFTIN1 probe FIGS. 1B and 1C), confirming a close relationship between rice osRAFTIN1 and wheat RAFTIN1 genes. In wheat as shown in FIG. 1C, there were 3 to 4 bands in the hexaploid (AABBDD genome), 2 in the tetraploid (AABB), 1 in one diploid (AA), and 1 to 2 in another diploid (DD). Careful comparison of the size of band(s) among different genomes strongly suggested that the RAFTIN1 gene family was most likely represented by one copy per A or B or D complement in wheats, the isolated taRAFTIN1a and taRAFTIN1b genes were likely from the AABB genome and the related gene in DD genome might consist of one HindIII restriction site in the coding region resulting two hybridization bands. This assumption was partially supported by the observation that a closely related genomic clone isolated from an AA genome wheat was closely related to taRAFTIN1a and the EST clone (GenBank accession no: BG274249, Anderson, O., 2001) obtained from a BB genome wheat was to taRAFTIN1b (data not shown).

RAFTIN1 is Specifically Expressed in the Anther

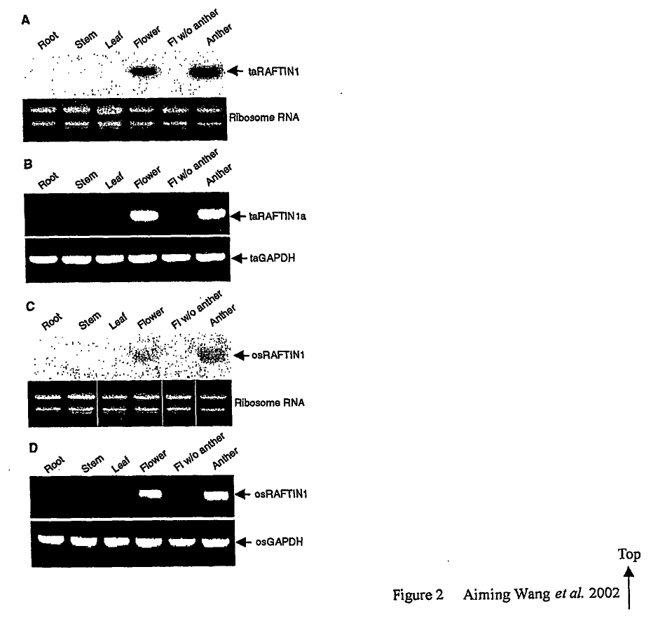

The expression pattern of RAFTIN1 in wheat and rice was determined by probing RNA blots with ORFs of taRAFTIN1a and osRAFTIN1. The taRAFTIN1a probe strongly hybridized only to RNA isolated from wheat young florescence or the anther but not from root, stem, leaf or the young florescence whose anther had been removed (FIG. 2A). Hybridization of transcripts isolated from corresponding tissues in rice using the osRAFTIN1 probe produced comparable results (FIG. 2C). In both species, the single hybridization band of ˜1.3 kb was consistent with the expected sizes of the RAFTIN1 cDNAs (FIGS. 2A and 2C). For further confirmation, more sensitive RT-PCR analysis was carried out with primer pairs designed to cover 2 partial exons containing 1 intron in-between in the genomic (for wheat RAFTIN1a) or containing 1 entire exon flanked with two intron (for rice osRAFTIN1) in-between in the genomic so as to discriminate amplicons of mRNA (875 bp for wheat; 441 bp for rice) and genomic DNA origin (974 bp for wheat; 703 bp for rice). A strong band with the predicted sizes of amplified cDNA fragments (875 bp for wheat; 441 bp for rice) was detected in the young florescence or anther samples starting from 22 cycles under the given conditions (see METHODS) but not (even up to 35 cycles) in the samples of root, stem, leaf or the florescence whose anther had been removed (FIGS. 2B and 2D). Thus, RAFTIN1 transcription was stringently restricted in the anther tissue.

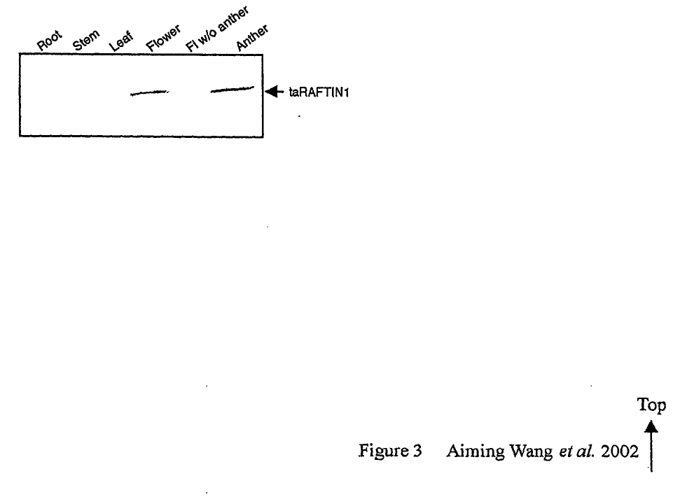

To detect RAFTIN1 proteins in planta, the polyclonal antibodies were raised against the N-terminal portion of the RAFTIN1a fusion protein over-expressed in E. coli. The purified antibodies were used for Western blot analysis of different wheat tissues. Consistently, RAFTIN1 proteins were only evident in wheat young florescence or the anther but not in root, stem, leaf or the young florescence whose anther had been removed (Fl w/o anther) (FIG. 3). The detected protein was ˜40 kDa in size, close to the predicted molecular weights (41.3 kDa and 38.3 kDa) for taRAFTIN1a and taRAFTIN1b. These results further confirmed the presence of taRAFTIN1a protein in the wheat anther.

RAFTIN1 Encodes a BURP Domain Containing Protein and RAFTIN1-Like Gene are not Present in the Arabidopsis Genome

Since the Arabidopsis genome has been sequenced and large knockout population is available, we searched the genomic sequence for RAFTIN1-like genes (www.arabidopsis.org). BLAST analysis did not identify in the model plant any known or hypothetical genes statistically significantly homologous to RAFTIN1 nt sequences. No overall significantly similar proteins were found in the Arabidopsis genome by BLASTP analyses. However, limited homology was shown between the C-terminal moiety (˜200 a.a.) of RAFTIN1, and the C-termini of 5 putative gene products, 36% identical to RD22 (a.a. 175-389) (Yamaguchi-Shinozaki & Shinozaki, 1993), 35% to an unknown protein (a.a. 86-277) (F13F21.25), 30% to an aromatic rich glycoprotein (a.a. 411-588) (F1707.9), 26% to a putative polygalacuronase isoenzyme 1 beta subunit (a.a. 311-619) (F508.31) and 27% to another polygalacturonase isoenzyme 1 beta subunit (a.a. 390-620) (T13D8.26). These conserved homologous regions were previously named BURP domain (see below). Searches for ESTs of these genes did not reveal any evidence that any EST of them was derived from anther or florescence tissues. Thus, apparently the rice and wheat anther-specific RAFTIN1 homologues were not present in the Arabidopsis genome.

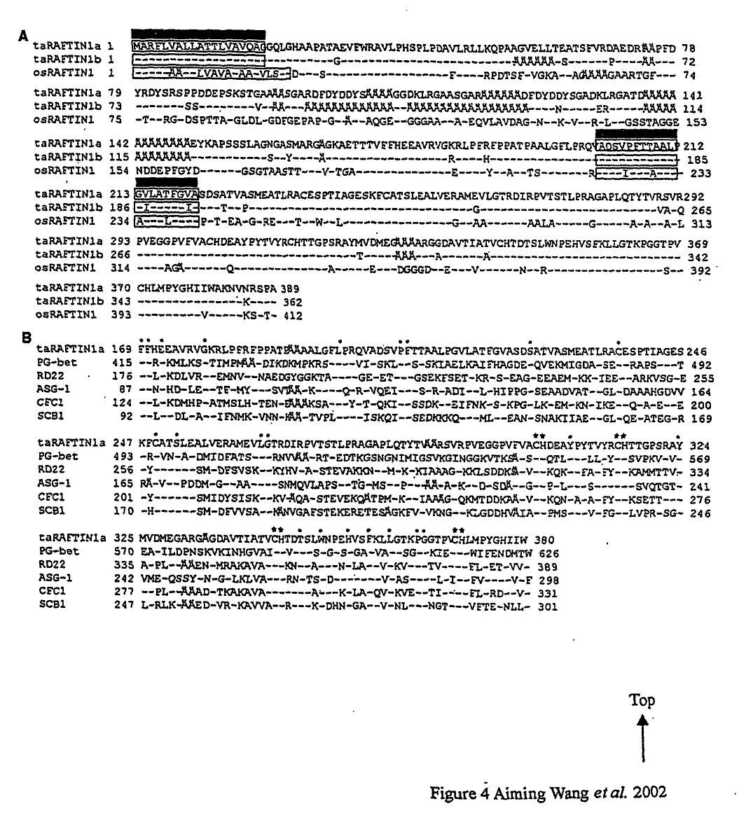

Further BLASTX searches of the public gene databases for RAFTIN1 homologous sequences corroborated that there were no overall similar genes documented in plants other than monocot cereals. In cereals, the significant matches were shown to 12 ESTs (˜600 bp) from anthers, young panicles or pre-anthesis spikes in bread wheat, a wild diploid wheat (Aegilopes speltoides, BB genome), rice, barley, rye, sorghum and maize. The GenBank accession numbers of these ESTs containing the longest 5′ sequences of each identical gene included BE40071 hexaploid wheat, available sequence identical to the 5′ terminal region of taRAFTIN1a cDNA), BE499238 (hexaploid wheat, identical to the 5′ terminal region of taRAFTIN1b), BG274249 (Aegilops speltoides, similar to the N-terminus of RAFTIN1), AU029260 (rice, identical to osRAFTIN1), AW562783 (maize, similar to the N-terminus of RAFTIN1), BE060637 (barley, similar to the N-terminus of RAFTIN1), BE636918 (rye, similar to the N-terminus of RAFTIN1) and BI140560 (sorghum, similar to the N-terminus of RAFTIN1). Domain searches revealed that there were two predicted transmembrane domains localized at the very N-terminal and the central regions, and a BURP domain at their C-terminal moiety (FIG. 4A). The BURP domain conserved in diverse plant proteins has recently been suggested to play an important and fundamental but uncharacterized role in plant (Hattori et al., 1998). The sequences of BURP domains, identified in this study and previously, were compared. Remarkably, all BURP domains conserved two phenylalanine residues (FF) at their N-termini of the domain and four repeated cysteine-histidine (CH) motifs in the consensus sequence CHX₁₀CHX₂₅₋₂₇CHX₂₄₋₂₅CH, where X represents any amino acid (FIG. 4B), similar with the previous finding (Hattori et al., 1998).

RAFTIN1 Transcripts are Found only in the Tapetum but RAFTIN1 Proteins are Present in the Tapetum and Microspore during the Rapid Growth Stages of Microspore

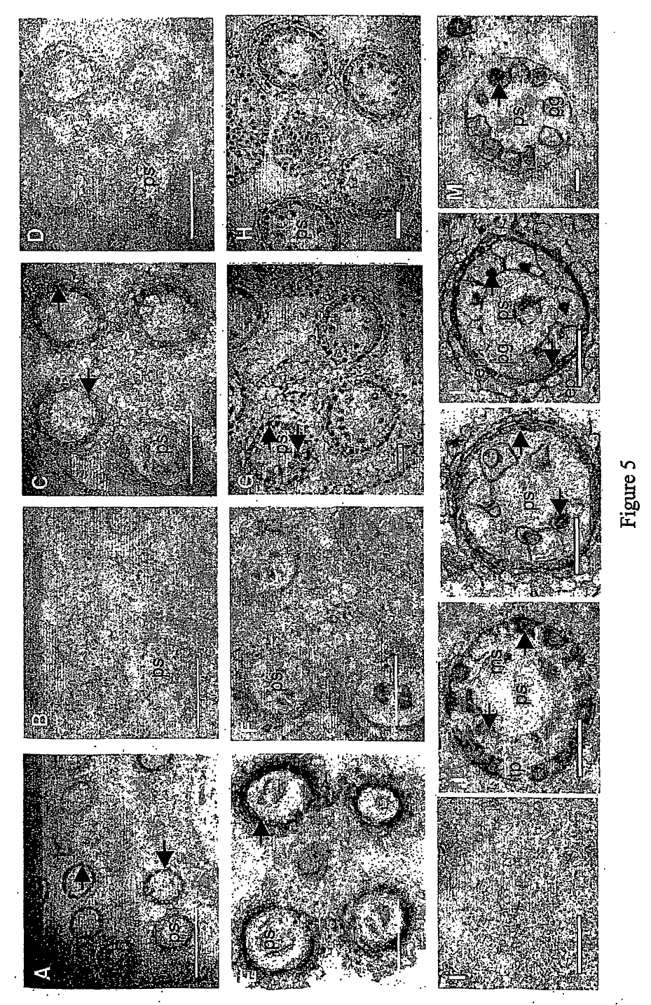

The anther encloses morphologically and functionally divergent tissues such as the anther wall (the epidermis, the endothecium and the middle layer), the tapetum, microspore and other supportive tissues. RAFTIN1 transcripts were localized by in situ RNA hybridization using a stretch of taRAFTIN1a or osRAFTIN1 antisense RNA synthesized in vitro as a probe. In both wheat and rice, the RAFTIN1 mRNA was found only in the tapetum but not in the ovary, the anther wall, microspore, the filament and other supportive tissues (FIGS. 5A, 5C and 5E). The hybridization signal was negative when the taRAFTIN1a or osRAFTIN1 sense RNA was used as a probe (FIGS. 5B, 5D and 5F). Thus, RAFTIN1 transcription was confined to the mono-layer cells of the tapetum surrounding microspore.

Cellular localization of RAFTIN1 proteins in the wheat anther was conducted by immunocytochemical analysis using the taRAFTIN1a polyclonal antibodies. Surprisingly, the positive signal for the presence of RAFTIN1 proteins was not only evident in the tapetum but also in microspore, albeit not detected in other tissues (FIG. 5G). The fidelity of this signal was corroborated by a pre-serum control (FIG. 5H). The detection of RAFTIN1 protein into the tapetum and microspore was inconsistent with tapetal localization of RAFTIN1 transcripts arising from the in situ RNA localization. This observation raised the possibility for translocation of RAFTIN1 proteins from tapetal cells to microspore.

To further explore the temporal expression pattern of RAFTIN1, the wheat floral sections at the various developing stages of microspore were probed with the purified taRAFTIN1a antibodies. RAFTIN1 proteins were not evident before the tetrad stage (FIG. 5I) and strongly detected in both the tapetum and the male gametophyte during post tetrad stages from the young free microspore stage to the vacuolated pollen grain stage when microspore underwent rapid expansion (FIGS. 5J and 5K). And weak signals were also found in the pollen maturation stages (5L and 5M). This spatio-temporal expression pattern of RAFTIN1 was further confirmed by histochemical GUS assay of the transgenic tobacco, Arabidopsis and rice plants transformed with an RAFTIN1 (taRAFTIN1a, taRAFTIN1b or osRAFTIN1) promoter: GUS chimeric gene (data not shown). These results indicated that RAFTIN1 transcription and translation were highly regulated during the male gametophyte development in wheat and rice.

RAFTIN1 Proteins are Subcellularly Localized to the Ubisch Body and the Microspore Extracellular Wall

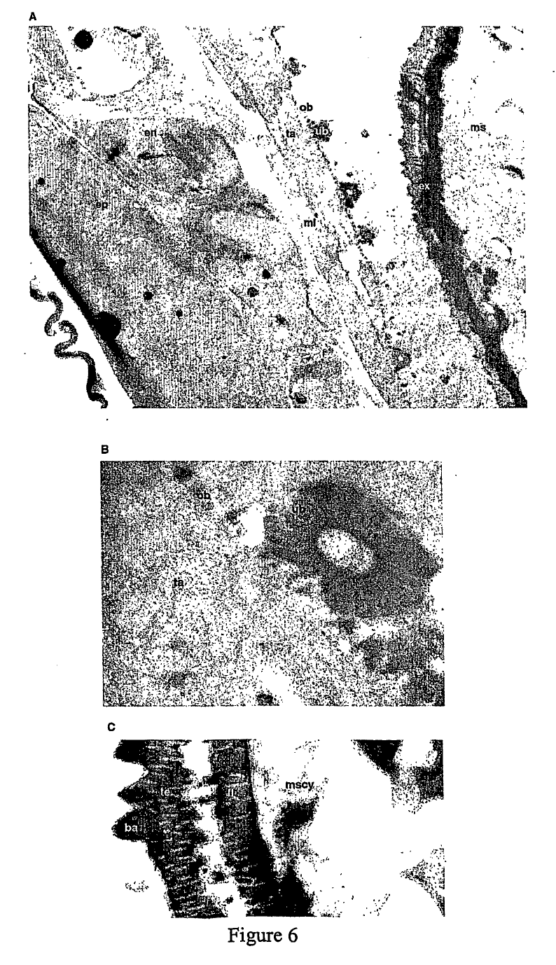

The taRAFTIN1a antibodies were used for subcellular localization of RAFTIN1 by immunoelectronic microscopy analysis. Consistent with the results obtained from the immunocytochemical assay, the antibodies exclusively detected the presence of RAFTIN1 in the tapetum and microspore (FIG. 6A). However, no significant labeling was found in the epidermis, the endothecium and other anther supportive tissues (FIG. 6A). No labeling was detected anywhere with the purified preimmune serum. Examinations of the positive cells immunolabeled with the anti-taRAFTIN1a serum revealed that gold labeling was most evident in the tapetal microsome-like structures, the Ubisch body, and the extracellular matrix (exinewall) of microspore but not in the orbicular wall or inside microspore (FIGS. 6B and 6C), further supporting that microspore was not likely to be the site for RAFTIN1 production. In the exinewall, gold particles were clearly shown in the tectum, the foot layer and bacules (FIG. 6C). Although lacking direct evidence, Ubisch bodies were previously hypothesized to be involved in transport of sporopollenin from the tapetum to the developing microspores (Huysmans et al., 1998). The localization of RAFTIN1 proteins in Ubisch bodies and the physical connection between the tapetum and the Ubisch body and between the Ubisch body and microspore supported that Ubisch bodies transported RAFTIN1, probably in a fashion similar to that by which sporopollenin is transported.

Intron-Spliced Hairpin RNA Effectively Reduces osRAFTIN1 Expression in Rice

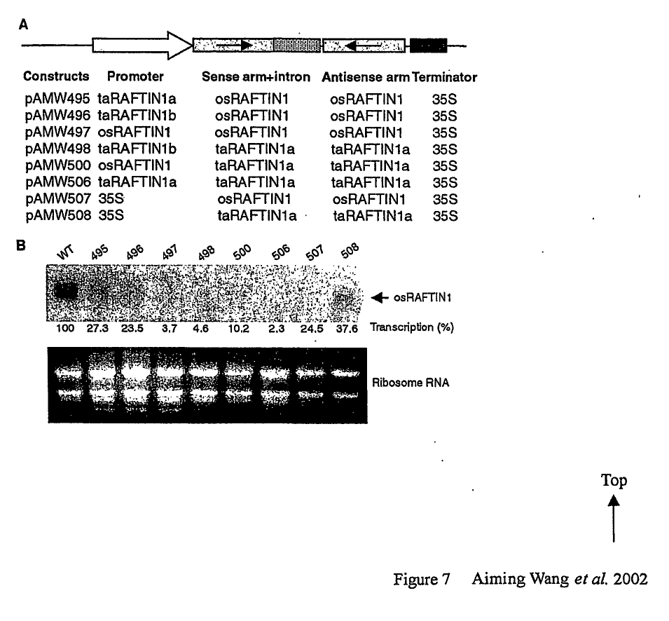

RAFTIN1 is a novel protein with no established function and is without a structural counterpart in Arabidopsis. Taking advantage of the findings that there is only one copy of RAFTIN1 in rice (in this study) and an intron-spliced hairpin RNA (ihpRNA) can efficiently induce sequence-specific gene silencing in plants (Smith et al., 2000), the transgenic approach was employed to silence osRAFTIN1 in rice to explore RAFTIN1 functions in planta. Eight rice transformation vectors harboring DNA sequences encoding ihpRNA homologous to osRAFTIN1 sequence under the control of taRAFTIN1a, taRAFTIN1b, osRAFTIN1 or 35S promoters were constructed (FIG. 7A); the chimeric genes were transformed into rice. Fifty-four lines transformed with these constructs and nine lines transformed with a selection marker gene (as a control) were recovered, showing resistance to the selection antibiotic, hygromycin and being positive in the PCR screening (data not shown). RNA isolated from the anthers of one randomly chosen representative line of each construct was used for Northern blot analysis to evaluate silencing efficacy (FIG. 7B). Of the transgenic lines examined, a line (497) containing osRAFTIN1 promoter, a partial genomic portion including an intron (at 3′ end) of osRAFTIN1, and the corresponding antisense sequence of osRAFTIN1 achieved the best silencing efficacy, with only 3.7% intact transcripts detected, in comparison with that in a control line (WT) containing the selection marker gene (FIG. 7B). The transgenic line with the poorest reduction of osRAFTIN1 expression was transformed with a 35S promoter followed with a taRAFTIN1a hairpin DNA, silencing 37.6% intact transcripts relative to WT. Thus, all the designed chimeric genes in the randomly picked lines could effectively induce osRAFTIN1 gene-specific silencing in the transgenic rice.

Down-Regulation of osRAFTIN1 Induces Male Sterility in the Transgenic Rice

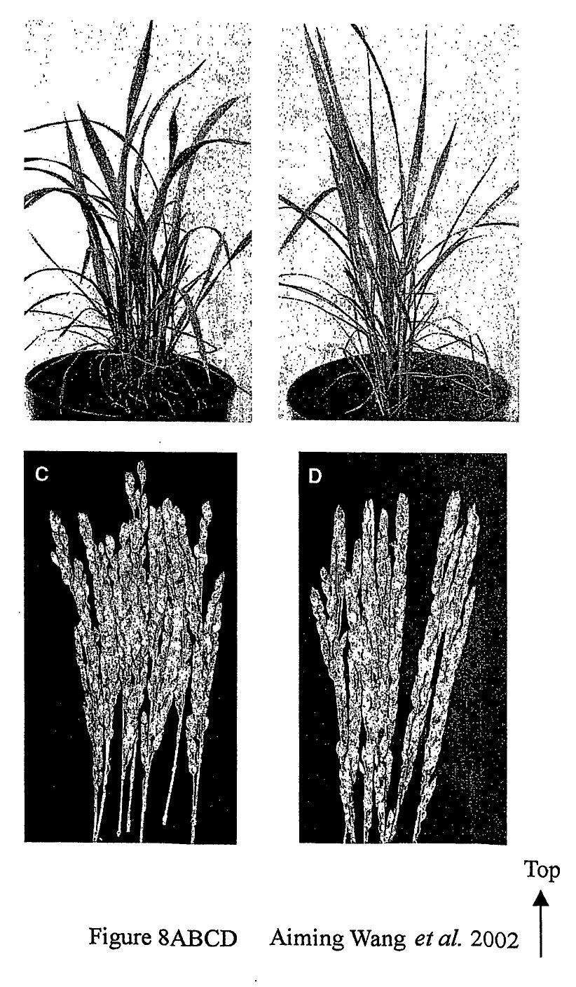

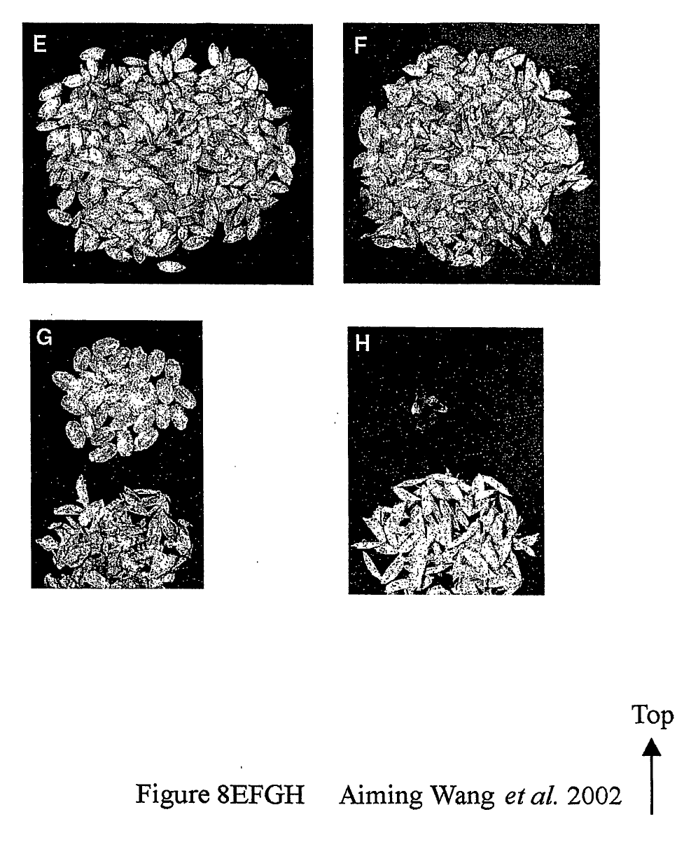

All the transgenic lines were morphologically observed for the consequences of down-regulation of osRAFTIN1 expression. The transgenic plants of the osRAFTIN1 silenced-lines and control lines showed similar tillering and leafing ability, similar leaf size, similar internode elongation and similar overall plant sizes (FIGS. 8A and 8B). Panicle initiation in all the lines occurred approximately 90-100 days post transplanting. All the lines had the similar unifloral spikelet subtended by two paleae lying between two glumes. All the flowers shared the same color, size and shape, and had apparently similar gynoecium with an ovary and two feathery stigmas. The androecium in the flower of all lines had six stamens and each of them had a filament and an anther. The mature panicle of the osRAFTIN1-silenced lines, however, was about 15-20% longer than that of the control lines (FIGS. 8C and 8D). The spikelet of the osRAFTIN1-silenced lines did not open and no anthesis took place at maturity even over long extended time (6 weeks) after the emergence of the panicle. No seeds or only a few seeds per plant (less than 1 seed per panicle) were produced in the osRAFTIN1-silenced lines, in contrast to more than 10 seeds per panicle in the control lines (FIGS. 8C, 8D, 8E, 8F, 8G and 8H). The palea in the osRAFTIN1-silenced lines kept green and did not turn yellow over an extended time (FIGS. 8E and 8F). The osRAFTIN1-silenced lines were crossed with pollen from a control line to test if the female organ was impaired. The successful production of hybrid seeds confirmed their normal ovary viability and function FIG. 8I). Therefore, the sterility in the osRAFTIN1-silenced lines arose from the male organ.

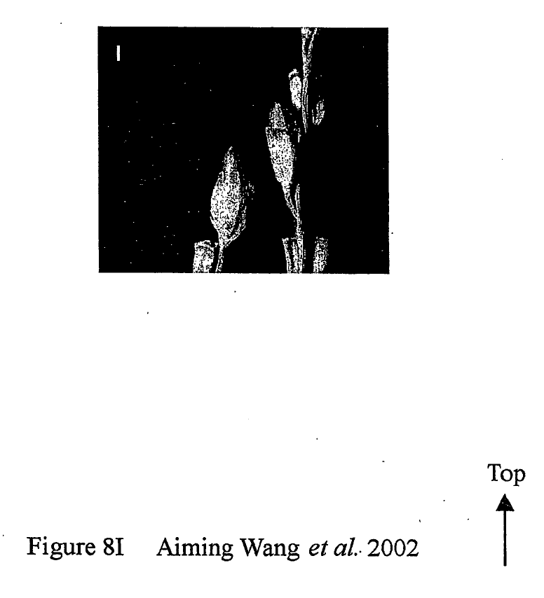

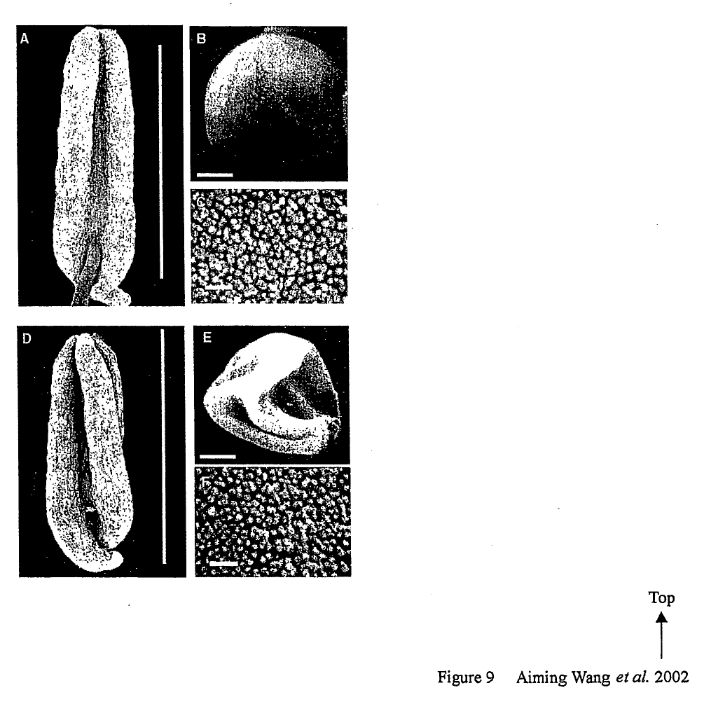

Accordingly, the anther in the osRAFTIN1-silenced lines was subjected to scanning electron microscopy. In the osRAFTIN1-silenced line, the mature anther was mostly malformed, non-dehiscent and 10-15% smaller in length (FIGS. 9A and 9D) and almost all the pollen grains were abnormal, showing signs of abortion (FIGS. 9B and 9E). But there was no detectable difference in the pollen surfaces (FIGS. 9C and 9F). In a germination assay, the germination rate of pollens in the osRAFTIN1-silenced lines was 4.7% while the germination rate of pollens in the control lines was 74.3%. Thus, in the osRAFTIN1 down-regulated lines (male sterile lines), vegetative growth and flower development were apparently normal prior to anthesis and the poor fertility was due to the abnormal development of the anther resulting from down-regulation of osRAFTIN1 expression.

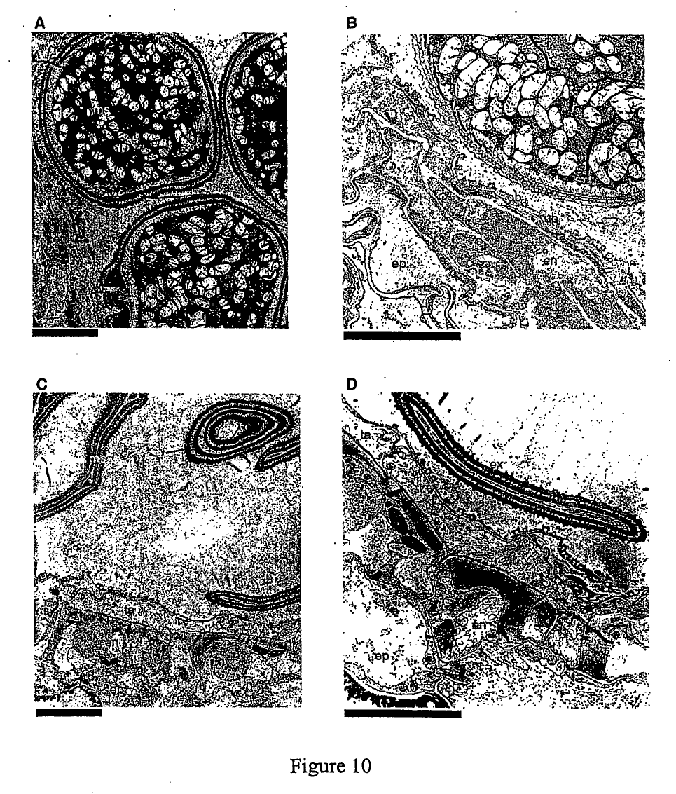

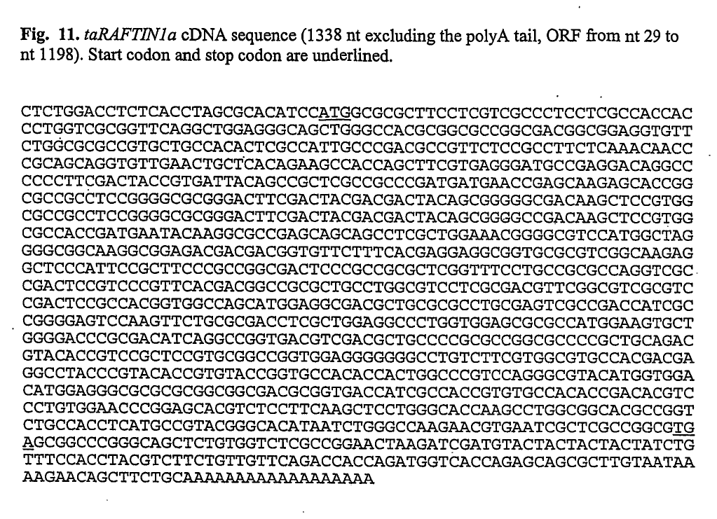

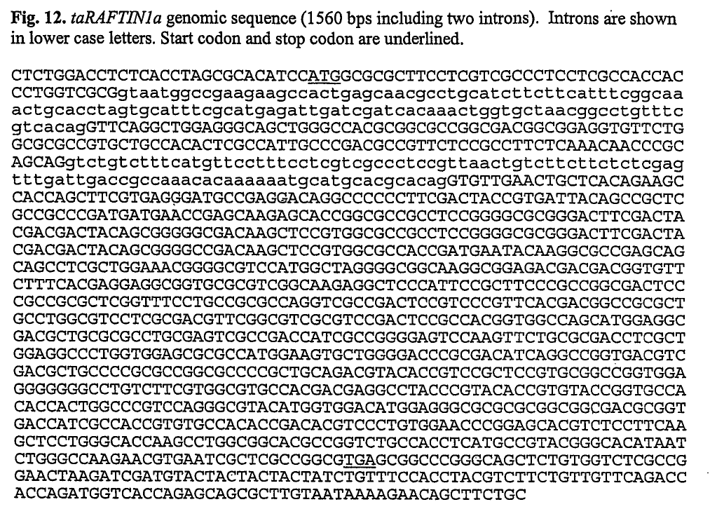

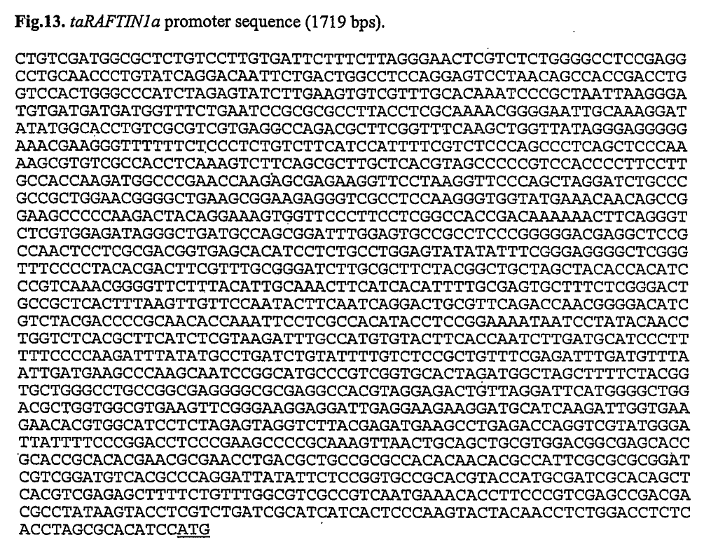

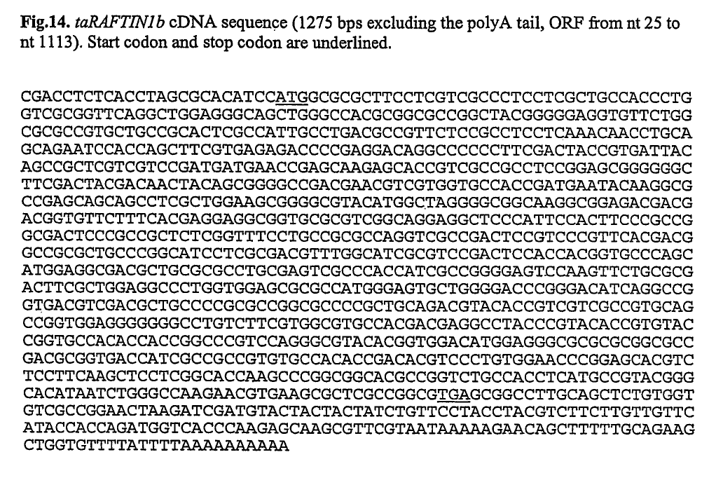

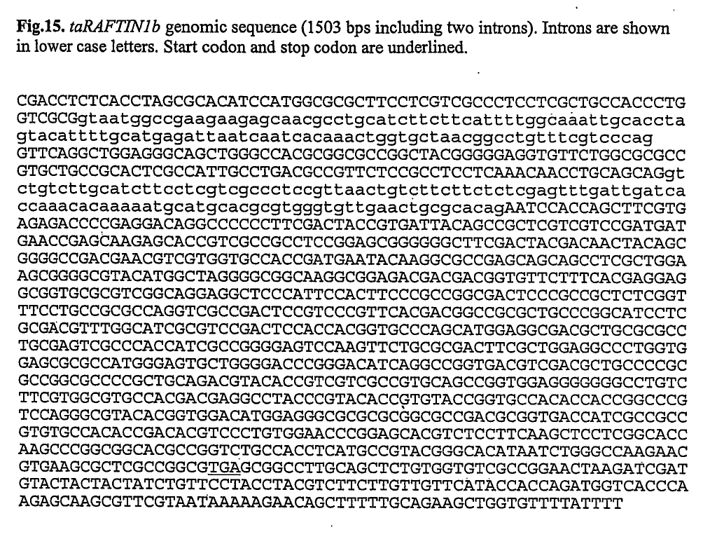

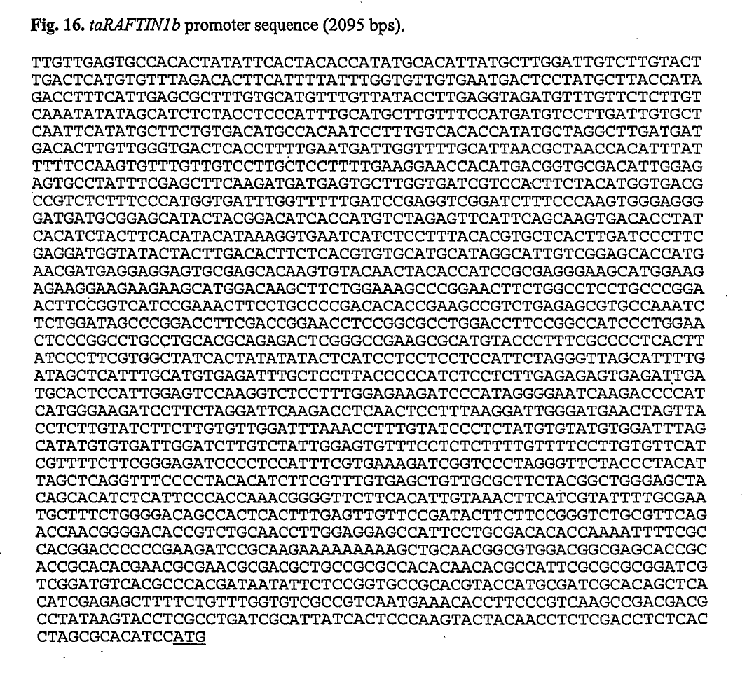

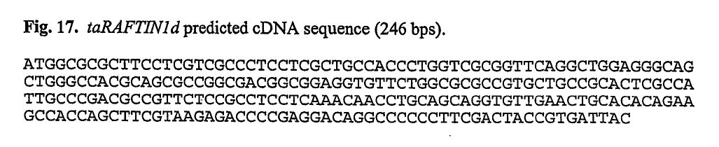

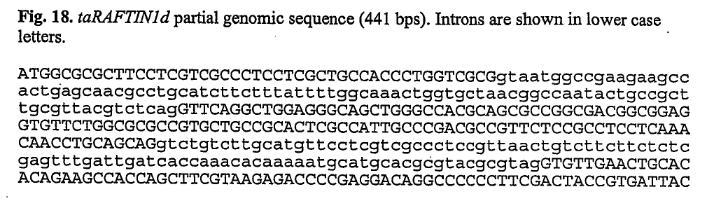

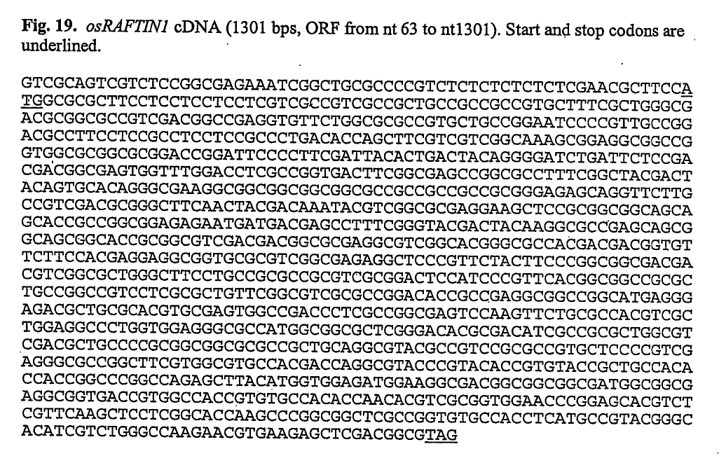

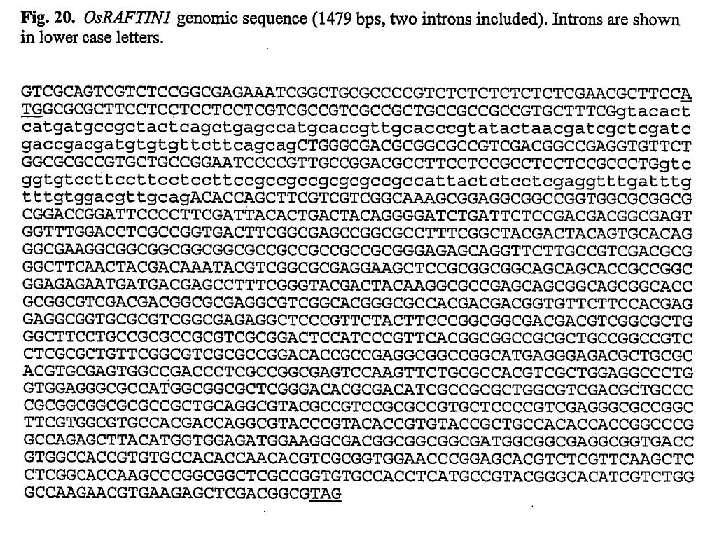

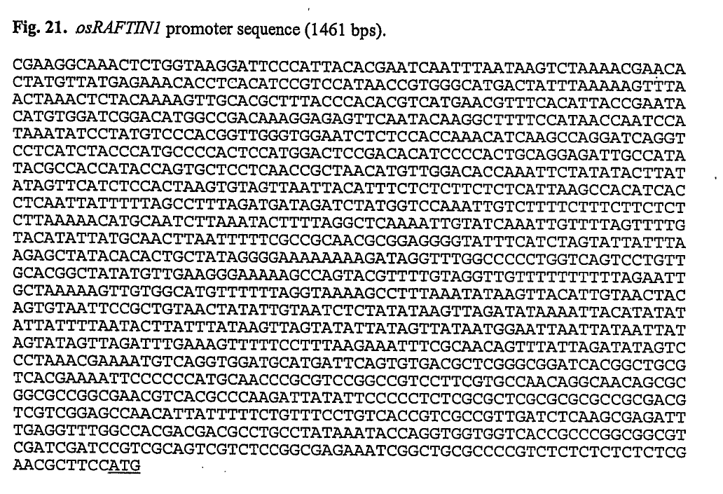

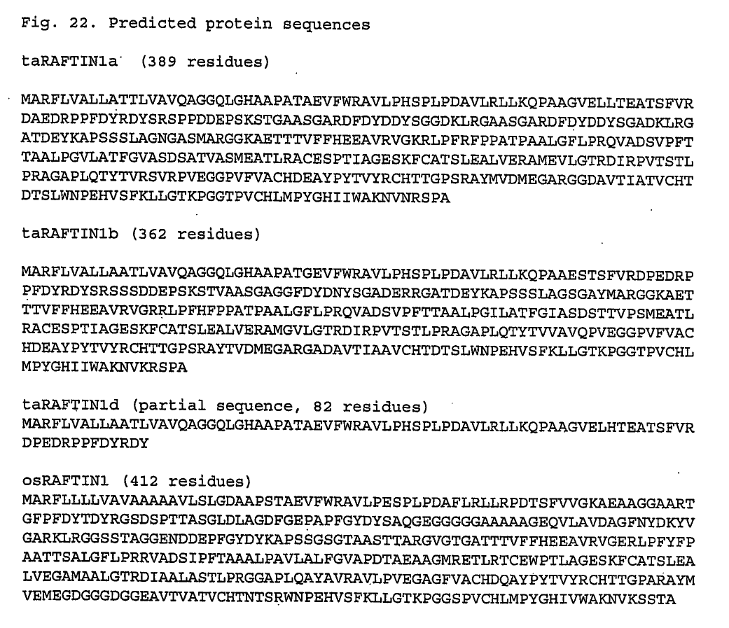

Transmission electron microscopy was applied to further examine anther development. In all the lines, the Ubisch body, the orbicular wall, and the exinewall were evident with no distinct difference (FIGS. 10A and 10B). In the control lines, the mature anther contained many round pollen grains rich in starch granules and other contents inside (FIG. 10A). In contrast, the anther of the osRAFTIN1-silenced lines contained mostly aborted, flat pollen grains with very little contents inside (FIG. 10B). The tapetum degeneration underwent well in the control lines with little tapetal remains, whereas in the osRAFTIN1-silenced lines, the tapetal degeneration was arrested apparently at the vacuolated microspore stage, leaving the half degenerated tapetum about 4 μm in thickness as clearly evident at the very mature stage when the endothecium wall was thickened (FIGS. 10C and 10D).

The cereal grains constituting more than 60% of total global agricultural production offer the major portion of the human diet. Understanding the molecular biology of anther development in cereal crops is of great importance for crop improvement. Based on studies in some model plants such as Arabidopsis and tobacco, it is known that the anther development is governed by a large number of genes with spatio-temporal expression specificity (Goldberg et al., 1993, Koltunow et al., 1990). It is estimated that in Arabidopsis there are approximately 3500 genes (13.7% of total predicted genes) which are specifically expressed in the anther and not in other floral and vegetative tissues (Sanders et al., 1999). Cereals have relatively larger genome sizes (hexaploid wheat 16000 Mb; rice 420-466 Mb) with more genes (rice: 32,000 to 55615 genes) (Goff et al., 2002, Yu et al., 2002) in comparison with Arabidopsis (125 Mb, 25498 genes) (The Arabidopsis Genome Initiative, 2000). Thus, it can be deduced that cereal anthers recruit more genes to execute microsporogenesis. Moreover, considering more than 50% rice genes do not have homologues in Arabidopsis, a considerable portion of anther-specific genes are probably unique to cereals, that may account for the morphologic and metabolic difference in the anther development between cereals and Arabidopsis. In this study we isolated the RAFTIN1 group of anther-tapetum specific genes, taRAFTIN1a, taRAFTIN1b and taRAFTIN1d in wheat and osRAFTIN1 in rice, that are apparently unique to grasses and not present in Arabidopsis or other eudicots. The localization of the RAFTIN protein onto the Ubisch body and the microspore exinewall raises the possibility that this protein is involved in transport of some macromolecules or their derivatives produced in the tapetum of cereals but not of Arabidopsis. Thus, study on RAFTIN1 assists in understanding the molecular genetics underlying the metabolic difference in anther development between cereals and Arabidopsis.

The Ubisch Body-Mediated Transport of RAFTIN1 and the Function of RAFTIN1

Ubisch bodies, or orbicules are the minute sporopollenin particles comprising acidic and neutral polysaccharides, proteins and unsaturated lipids that line the inner surface of most secretory tapeta (El-Ghazaly & Jensen, 1986, El-Ghazaly & Jensen, 1987, Huysmans et al., 1998, Suarez-Cervera et al., 1995). Although discovered more than a century ago, Ubisch bodies, in terms of their structure, origin, development and function, remain mysterious. Based on cytological studies mainly through electron microscopy, it is believed that Ubisch bodies originate from so-called “grey bodies” or “globular bodies” (pro-orbicule; pro-Ubisch bodies) derived from endoplasmic reticulum in the cytoplasm of the tapetum as early as the meiosis and tetrad stages (El-Ghazaly & Jensen, 1986, El-Ghazaly & Jensen, 1987, Huysmans et al., 1998, Suarez-Cervera et al., 1995). Approaching the plasma membrane, pro-Ubisch bodies are bound by membranes (El-Ghazaly & Jensen, 1986). At the free microspore stage, pro-Ubisch bodies, upon fusion of the plasma membranes, are released from but connected with the plasma membrane by a layer of microfibril. Along with the coating and accumulation of sporopollenin on the surface of pro-Ubisch bodies, they mature to Ubisch bodies. At the same time, sporopollenin is deposited between and beneath Ubisch bodies to form an orbicular wall (El-Ghazaly & Jensen, 1986). In the previous studies, functions hypothesized for the Ubisch body include transport of sporopollenin, temporary packing of sensitive material for transport through locular sap, by-products of tapetal cell metabolism, association with pollen dispersal, degradation of tapetal cells, and prevention against osmosis and collapse of developing microspores (Huysmans et al., 1998).

In this study, we found that RAFTIN1 proteins were localized to the tapetum, the Ubisch body and the microspore exinewall, but not on the orbicular walls and inside microspore. Moreover, RAFTIN1 transcript was only evident in the tapetum. Since Ubisch bodies are physically located between the plasma membrane of the tapetum and the exinewall of microspore, it is therefore conceivable that through the Ubisch body, RAFTIN1 is transported from the tapetum to microspore. RAFTIN1 proteins were not detected until the microspore stage, suggesting that the deposition of RAFTIN1 into the Ubisch body and further onto the microspore exinewall is probably concurrent with that of sporopollenin. Silencing RAFTIN1 expression did not disrupt or discernibly change the structure of Ubisch bodies and the microspore exinewall, indicating RAFTIN1 is not required for building-up the basic skeleton of the Ubisch body and the microspore exinewall. However, we found that in the RAFTIN1-silenced lines, the tapetal degeneration was clearly retarded, and microspore was smaller and contained much less contents. One possible explanation is that RAFTIN1 is required for the degradation of tapetal cells and the failure to such degradation terminates the nutrient and metabolite release from the tapetum to the locule leading developing microspore starving to abortion. However, this assumption is not in agreement with the following findings: RAFTIN1 proteins, though synthesized in the tapetum, are transported outside of the tapetum; RAFTIN1 does not have any known domains for such a possible function; and over-expression of RAFTIN1 does not induce cell degradation in a model plant. Therefore, RAFTIN1 is not likely a candidate protein that involves the programmed cell death of the tapetum. Alternatively, RAFTIN1 probably directly or indirectly regulates transport of certain metabolites which are rich in the tapetum of cereal crops, such as acid polysaccharides, neutral polysaccharides or their derivatives. Cereal pollen grains accumulate large amounts of starch granules (this study; (Bedinger, 1992) (Zhang et al., 2001). During microspore development, carbohydrate metabolism takes place very actively in the tapetum and in the surrounding supportive tissues as well as in microspores. In wheat, substantial amounts of acidic polysaccharides, proteins, neutral polysaccharides and to a lesser extent unsaturated lipids are found in the Ubisch body and the microspore exinewall over a long period during microspore development (El-Ghazaly & Jensen, 1987). Disturbance in carbohydrate metabolism induces male sterility (Dorion et al., 1996, Lalonde et al., 1997, Zhang et al., 2001). Interestingly, in crucifers and other entomophilous species where no RAFTIN1-like ESTs or genes have been documented, it is lipids not carbohydrates that are major products of the tapetum (Piffanelli et al., 1997), implying a difference between major tapetal metabolism of cereals and that of entomophilous species. The RAFTIN1-less Ubisch body and microspore exinewall probably is incompetent to transport these metabolites from the degenerating tapetum to microspores, which disconnects the nutrient supply-pipe to microspores leading to pollen abortion and, in turn, slows down the tapetal degeneration.

Molecular Hallmarks of BURP Domain-Containing Proteins and their Multi-Functional Roles

The C-terminal moiety of RAFTIN1 shares extensive sequence homology with a BURP domain that has been found only in the plant kingdom. These include RD22 (a gene responsive to dehydration stress, high salt or ABA induction) from Arabidopsis (Yamaguchi-Shinozaki & Shinozaki, 1993), A2-134 (or ASG-1) in the developing embryo of apomictic guinea grass (Panicum maximum) (Chen et al., 1999), the β subunit of polygalacuronase isoenzyme 1 (PG1β) in the developing fruit of tomato (Zheng et al., 1992), USP showing seed-specific in fava bean (Vicia faba) (Bäumlein et al., 1991), BNM2 (a gene expressed during the induction of microspore embryogenesis) in rape (Brassica napus) (Hattori et al., 1998), ADR6 (an auxin down-regulated gene) in soybean (Datta et al., 1993), SALI 3-2 (an aluminum up-regulated gene) from soybean roots (Ragland & Soliman, 1997) and some ESTs from Prunus persica (GenBank accession no: AAL26909), from cotton fiber cells (GenBank accession no: AAL67991), and from soybean seed coats (GenBank accession no: AAL76058; gene named SCB1). Thus, in spite of highly conserved primary structural feature within their C-terminal BURP domains, the BURP domain proteins are expressed in divergent tissues and under various conditions. Nevertheless, these proteins are expressed in the tissues either under stress (chemicals, ABA, high salts, dehydration, auxin-down regulation), or during development (the anther, fruit and seed), which are undergoing active biodegradation and biosynthesis metabolism and probably intercellular metabolite movement. Alignments of the BURP domains confirm the previous finding that there is a consensus sequence, CHX₁₀CHX₂₅₋₂₇CHX₂₄₋₂₅CH containing four repeated cysteine-histidine (CH) motifs located at C-termini (Hattori et al., 1998). These conserved motifs are the hallmarks of the BURP domains and are probably involved in the formation of disulfide bond intramolecularly for the proper protein folding or more likely intermolecularly for anchoring of the protein onto specific sites of the cell wall. This later notion is supported by observations that all the BURP domain proteins are only found in the plant kingdom and that the two only characterized ones, i.e., RAFTIN1 (this study) and PG1β (Zheng et al., 1992) have been subcellularly localized into the Ubisch body and the microspore exinewall, and the cell wall, respectively. The N-terminal moiety of the BURP domain proteins is highly divergent. This divergence may reflect their multi-functionality. Thus, PG1β, a 69 kDa non-catalytic fruit-specific cell wall glycoprotein that is proposed to play a role in the localization, immobilization or activation of the polygalacturonase enzyme complex within the cell wall, may use its C-terminal BURP domain for cell wall attachment, and its N-terminal domains for association with the catalytic subunit and regulation of its activity. In contrast, RAFTIN1 is anchored to the Ubisch body and the microspore exinewall by its BURP domain and its N-terminal moiety regulates metabolite transport. Further characterization of other BURP domain proteins will assist in unraveling the functional mystery of the BURP domain proteins, perhaps the key role of the biological process in anther development.

The presence of RAFTIN genes has been demonstrated in a number of plant species, especially those belonging to monocots. Thus, it appears that this gene is likely to be present in other monocots including other cereals and grasses. Silencing or knocking-out of RAFTIN will find utility in breeding programs where male sterile lines are required. Furthermore, this technology can be used to prevent flow of transgenic pollens from elite transgenic lines, e.g., herbicide resistance lines, to wild plant species by silencing or knocking-out RAFTIN genes.

Methods

Plant Materials

Hexaploid spring wheat (Triticum aestivum L. cv. AC Karma, AABBDD), tetraploid wheat (T. turgidum L. cv. Sceptre, AABB), two diploid wheat species (T. urartu, ssp. nigrum, AA; T. tauschii L. china, DD), were obtained as described previously (Wang et al., 2002) and rice (Oryza sativa L. japonica var. nipponbare) was obtained from National Institute of Agrobiological Resources, Japan, and were grown in a greenhouse.

Isolation of taRAFTIN1a, taRAFTIN1b and osRAFTIN1 cDNAs, and their Corresponding Genomic Sequences

A cDNA clone (A71) was obtained from a wheat anther cDNA library constructed. previously (Wang et al., 2002). As BLASTN or BLASTX searches of 5′ sequence of A71 against the Arabidopsis genome did not identify any A71-like sequence, the insert of clone A71 was completely sequenced. The 5′ region of the predicted open reading frame (ORF) was amplified by PCR with primers OL3044 (5′TGCCACACTCGCCATTG3′) (SEQ ID NO: 1) and OL3045 (5′TTTCCAGCGAGGCTGCT3) (SEQ ID NO: 2). The resulting 346 bp DNA fragment was used as a probe to screen ˜500,000 clones of the anther cDNA library. The phagemids from the 26 positive plaques, excised in vivo using the ExAssist/SOLR system (Stratagene), were sequenced. Based on sequences, these cDNAs were placed into two groups, and the longest of each group was named taRAFTIN1a and taRAFTIN1b accordingly.

The entire osRAFTIN1 ORF was cloned by RT-PCR using primers OL4382 (5′CGGGGTACCGAACGCTTCCATGGCGCGCT3′ (SEQ ID NO: 3), KpnI site underlined, start codon ATG in italic) and OL4383 (5′GCTCTAGAGCTTCTACGCCCGTCGAGCTC3′ (SEQ ID NO: 4), XbaI site underlined, the codon in italic is complementary to the stop codon TAG). The cDNA was directionally cloned into the KpnI-XbaI sites of plasmid pBluescript SK (Stratagene).

The genomic counterparts of taRAFTIN1a, taRAFTIN1b and osRAFTIN1 were obtained by PCR of the genomic DNA, cloned into the T/A vector (Invitrogen, Carlsbad, Calif., USA) and sequenced.

Oligonucleotide Synthesis, DNA Sequencing and Sequence Analysis

Oligonucleotide synthesis and DNA sequencing were carried out by the DNA Technology Unit of the Plant Biotechnology Institute. DNA sequences were assembled and analyzed using Lasergene software (DNASTAR Inc., Madison, Wis., USA), FASTA (www.ebi.ac.uk/fasta3/), BLAST 2 and BLAST (www.ncbi.nlm.nih.gov).

DNA and RNA Gel Blot Analysis

Genomic DNA was isolated from leaves as described (Wang et al., 2002). DNA was digested with appropriate restriction enzymes, fractionated by electrophoresis on a 0.8% agarose gel, and transferred onto a Hybond N⁺ membrane (Amersham, Baie d'Urfe, Quebec, Canada). The methods for nucleic acid isolation, blotting, ³²P-labeling of probes and hybridization were as described (Wang et al., 2002). The entire ORFs of taRAFTIN1a and osRAFTIN1 retrieved by PCR were used as probes for hybridization. Hybridization and subsequent washing conditions were essentially as described (Wang et al., 2002).

RT-PCR Analysis of RAFTIN1 Gene Expression

First strand cDNA was generated in a 20 μl reaction containing 5 μg of total RNA isolated from appropriate wheat/rice tissues, 0.5 μg oligo (dT)₁₈, 20 units of SUPERSCRIPT™ II RNase H⁻ Reverse Transcriptase (Invitrogen) according to the supplier's instruction. One hundred-fifty ng RNA-derived cDNA was used for a 100-μl PCR reaction in the presence of 10 units of Taq DNA polymerase (Amersham). Primers OL3044 and OL3073 (5′CTCCATGTCCACCATGTA3′) (SEQ ID NO: 5) were used for amplification of an 875-bp fragment at the 5′ coding region of wheat taRAFTIN1a cDNA, whereas primers OL3148 (5′CGACGTATTTGTCGTAGT3′) (SEQ ID NO: 6) and OL3815 (5′TCTCGAACGCTTCCATG3′) (SEQ ID NO: 7) were targeted at a 441-bp cDNA immediately from the putative start codon of rice osRAFTIN1. Primers OL4556 (5′TCGAGCTCGTCGCCGTCA3′) (SEQ ID NO: 8) and OL4557 (5′GCAGCACCAGTGCTGCTG3′) (SEQ ID NO: 9) binding to cDNA of a house-keeping gene, glyceraldehyde-3-phosphate dehydrogenase (GAPDH) were used as a control. All PCR was carried out with a Techne Genius thermocycler (Duxford, Cambridge, UK) as indicated: 35 cycles of 94° C., 30 sec; 56° C., 30 sec; and 72° C., 1 min; finally a 10-min extension at 72° C. Five μl of the reaction was used for agarose gel analysis.

Production and Purification of RAFTIN1 Polyclonal Antibodies

The 5′ region of taRAFTIN1a ORF was amplified by PCR using a BamHI site-containing 5′ primer (5′CGGGATCCGCGCTTCCTCGTCGC3′ (SEQ ID NO: 10), BamHI site underlined) and an EcoRI site-containing 3′ primer (5′GGAATTCTCACGCCGGCGAGCGATT3′ (SEQ ID NO: 11), EcoRI site underlined) and cloned in-frame into the BamH1-EcoRI sites of plasmid pTrxFus (Invitrogen, Carlsbad, Calif., USA) to yield plasmid pAMWthio-A71. The fusion protein produced in E. coli strain GI724 (Invitrogen) hosting plasmid pAMWthio-A71 was purified (Wang & Sanfacon, 2000) and used for immunizing rabbits (by the staff at Veterinary Infectious Diseases Organization, University of Saskatchewan, Saskatoon, Canada). Antiserum IgG was initially purified as described (Wang et al., 1999) and further purified using Affi-Gel 10 Gel (Bio-RAD, Mississauga, Ontario, Canada) following supplier's instructions.

Protein Extraction, Subcellular Localization and Western Blotting Analysis

For total protein extraction, different wheat tissues (200 mg each) were homogenized with 1 ml extraction buffer (50 mM Tris-HCl, pH 8.0, 150 mM NaCl, 1% Triton X-100, 1% sodium deoxycholate, 10 mM β-mercaptoethanol, 1 mM phenylmethylsulfonyl fluoride (PMSF) and 1 mM ethylenediaminetetraacetic acid (EDTA)). The homogenate was centrifuged at 10,000×g at 4° C. for 10 min. The supernatant was used for protein blotting analysis.

For protein subcellular location, 1 g developing wheat anthers were homogenized with 2 ml PSL buffer (protein subcellular localization buffer: 100 mM Tris-HCl, pH 7.5, 5 mM MgCk₂, 10 mM KCl, 10% glycerol, 0.4 M sucrose, 10 mM β-mercaptoethanol, and 1 mM PMSF) on ice. The homogenate was filtered through an 80 mesh nylon cloth. The insoluble materials containing cell wall debris, after washing 3 times with PSL buffer plus 1% Triton X-100, were obtained as the cell wall fraction. The filtrate was centrifuged at 1,000×g for 10 min at 4° C. The pellet rich in nucleus was called the nucleus fraction. The supernatant was centrifuged at 30,000×g for 30 min at 4° C. The resulting pellet mainly containing membranous materials was the membrane-binding fraction and the supernatant containing the soluble protein was the cytosol fraction. All fractions were diluted to 2 ml with PSL buffer. Equal volume of each fraction was used for western blot analysis.

For immunoblot analysis, proteins were separated by denaturing sodium dodecyl sulfate-polyacrylamide gel electrophoresis (SDS-PAGE) (Laemmli, 1970) and transferred onto a polyvinylidene fluoride (PVDF) membrane using a Bio-Rad miniblotter at 125 V for 2 hr in a Tris/MeoH/glycine buffer (25 mM Tris-HCl, pH 8.3, 20% (v/v) methanol, 192 mM glycine). The membrane was incubated in a blocking solution containing 3% bovine serum albumin (BSA) in TBS buffer (20 mM Tris-HCl, pH 7.5, 500 mM NaCl) with gentle agitation for 30 min, and then washed twice with TBS buffer for 5 min each. After incubation with the first antibodies in incubation buffer (1% BSA and 0.05% Tween-20 in TBS) for 1 hr, the filter was washed with washing buffer (0.05% Tween-20 in TBS) for 5 min three times. Secondary antibodies conjugated with alkaline phosphatase (goat anti-rabbit IgG; Bio-RaD) (1:2000 diluted with incubation buffer) were incubated with the filter for 1 hr. The filter was washed three times with washing buffer and twice with alkaline phosphatase buffer (AP buffer: 100 mM Tris-HCl, pH 9.5, 100 mM NaCl, 50 mM MgCl₂). The antibody-protein complex was visualized with enzyme substrates, BCIP (bromochloroindolyl phosphate; Bio-Rad) and NBT (nitro blue tetzzolium; Bio-Rad) in AP buffer. The reaction was stopped by addition of excess stop buffer (20 mM Tris-HCl, pH 7.5, 5 mM EDTA). All steps were performed at RT except otherwise stated.

In Situ RNA Hybridization and Immunocytochemistry

Rice and hexaploid wheat flower sections, in situ RNA hybridization and immunocytochemistry were performed as described (Wan et al., 2002, Wang et al., 2002).

Promoter Isolation

The upstream regulatory regions of taRAFTIN1a and taRAFTIN1b coding regions were isolated from the hexaploid wheat cultivar Karma (genetic complements: AABBDD) using a Universal GenomeWalker™ Kit (Clontech, Palo Alto, Calif.). Two nested reverse primers, OL3070 (5′TCCAGCCTGAACCGCGACCAGGGTGGT3′) (SEQ ID NO: 12) and OL3071 (5′GTGGTGGCG-AGGAGGGCGACGAGGAA3′) (SEQ ID NO: 13) were used for the first and second PCR. The resulting two fragments of 1.7 kb for taRAFTIN1a and 2.1 kb for taRAFTIN1b were cloned into a T/A vector (Invitrogen). The inserts were completely sequenced.

A DNA fragment 1458 bp upstream of the predicted start codon for osRAFTIN1 ORF was retrieved directly by PCR of the genomic DNA using primers OL3079 (5′CGAAGGACTCTGGT3′) (SEQ ID NO: 14) and OL3080 (5′CATGGAAGCGTTCGAGA3′) (SEQ ID NO: 15) and cloned into a T/A vector (Invitrogen).

Transformation Vector Construction and Plant Transformation

For promoter analysis in tobacco and Arabidopsis, cloned promoters were retrieved using appropriate sets of primers: OL3861 (5′CCCAAGCTTCTGTCGATGGCGCTCTGT3′ (SEQ ID NO: 16), HindIII site underlined) and OL3862 (5′CGGGATCCGATGTGCGCTAGGTGAGA3′ (SEQ ID NO: 17), BamHI site underlined) for amplification of taRAFTIN1a upstream regulatory region (1716 nt upstream of start codon), OL3863 (5′CAAGCTTCTAGACTTGTTGAGTGCCACACT3′ (SEQ ID NO: 18), HindIII site underlined) and OL3862 for taRAFTIN1b (2092 nt upstream of start codon), OL3142 (5′CCCAAGCTTTACCCACACGTCATGA (SEQ ID NO: 19), HindIII site underlined) and OL3143 (5′CGGGATCCCATGGAAGCGTTCGAGA3′ (SEQ ID NO: 20), BamHI site underlined) for osRAFTIN1 (1309 nt upstream of start codon). The PCR products were restricted and ligated into the HindIII-BamHI sites of the modified plasmid pAMW477 (Wang et al., 2002) in which the TAA1a coding region had been replaced with a GUS gene from plasmid pRD420 (Datla et al., 1992) to generate plasmid pAMW484 (taRAFTIN1a::GUS chimeric gene), pAMW483 (taRAFTIN1b::GUS), and pAMW486 (osRAFTIN1::GUS). Genetic transformation of Nicotiana tabacum cv. Xanthi and Arabidopsis thaliana ecotype Col-0 were essentially as described (Clough & Bent, 1998, Wang et al., 2002). For promoter analysis in rice, the HindIII-KpnI fragment of plasmids pAMW484, pAMW483 and pAMW486 containing the RAFTIN1::GUS cassette were individually co-ligated with an HindIII-KpnI fragment (35S::hph cassette) of plasmid pBShph (R. Datla, Plant Biotechnology Institute, National Research Council of Canada, Canada) to produce plasmids pAMW 499, pAMW494 and pAMW501, respectively.

ihpRNA intermediate clone pAMW487 containing RAFTIN1a promoter, complementary sequence of osRAFTIN1 cDNA (nt 159 from start codon to nt 415), its corresponding sense genomic DNA sequence with additional 114 nt of upstream genomic sequence including an 82 nt intron, and a 35S terminator hereafter named RAFTIN1::hp-osRAFTIN1) was constructed by co-ligation of a BamHI-SpeI restricted PCR fragment of osRAFTIN1 cDNA amplified with OL3888 (5′CGGGATCCGACGTATTTGTCGTAGT3′ (SEQ ID NO: 21), BamHI site underlined) and OL3889 (5′GGACTAGTCAGCTTCGTCGTCGGCA3′ (SEQ ID NO: 22), SpeI site underlined) and an XbaI-EcoRI restricted PCR fragment of osRAFTIN1 genomic DNA with OL3886 (5′GCTCTAGACGCCTTCCTCCGCCT3′ (SEQ ID NO: 23), XbaI site underlined) and OL3887 (5′GGAATTCGACGTATTTGTCGTAGT3′ (SEQ ID NO: 24), EcoRI site underlined) into BamHI-EcoRI sites of plasmid pAMW484. Similar strategy was used to make clone pAMW488 containing taRAFTIN1b::hp-osRAFTIN1, clone pAMW489 containing osRAFTIN1::hp-osRAFTIN1, and clone pAMW503 (35S::hp-osRAFTIN1).

Clone pAMW491 containing taRAFTIN1b promoter, complementary sequence of taRAFTIN1a cDNA (nt 181 from start codon to nt 544), its corresponding sense genomic sequence with additional 148 nt of upstream genomic sequence including a 99 nt intron), and a 35S terminator (hereafter named taRAFTIN1b::hp-taRAFTIN1a) was created by coligation of a BamHI-SpeI restricted PCR fragment of taRAFTIN1a cDNA amplified with OL3892 (5′CGGGATCCTGGGAGCCTCTTGCCGA3′ (SEQ ID NO: 25), BamHI site underlined) and OL3893 (5′GGACTAGTCACAGAAGCCACCAGCT3′ (SEQ ID NO: 26), SpeI site underlined) and an XbaI-EcoRI restricted PCR fragment of taRAFTIN1a genomic DNA with OL3890 (5′GCTCTAGACGCCGTTCTCCGCCT3′ (SEQ ID NO: 27), XbaI site underlined) and OL3891 (5′GGAATTCTGGGAGCCTGTTGCCGA3′ (SEQ ID NO: 28), EcoRI site underlined) into BamHI-EcoRI sites of plasmid pAMW485. Similar strategy was employed to generate subclones pAMW492 containing osRAFTIN1::hp-taRAFTIN1a and pAMW504 containing 35S::hp-taRAFTIN1a.

Subclone pAMW502 containing taRAFTIN1a::hp-taRAFTIN1a was constructed by insertion of the small fragment from BamHI-KpnI double digested pAMW491 into the corresponding sites of pAMW484. The small fragment of clones pAMW487 (3.1 kb), pAMW488 (2.9 kb), pAMW 489, pAMW491 (2.8 kb), pAMW492 (3.0 kb), pAMW502 (3.0 kb), pAMW503 (2.8 kb) and pAMW504 (2.9 kb) double-digested with HindIII-KpnI was ligated with the small 1.7 kb HindIII-EcoRI fragment (35S::hph cassette) of plasmid pBShph and plasmid pHS723 (Huang et al., 2000) digested with KpnI and EcoRI to obtain ihpRNA transformation vectors pAMW495, pAMW496, pAMW497, pAMW498, pAMW500, pAMW506, pAMW507 and pAMW508, respectively. Rice transformation was carried out following the published protocol (Chen et al., 1998).

Pollen Viability Assay

Pollen from mature anthers of the osRAFTIN1-silenced lines or the control lines was stained with 1% aniline blue in lactophenol. The viability of pollen was calculated based on over 300 pollen grains.

Scanning Electron Microscopy (SEM), Transmission Electron Microscopy (TEM) and Immunoelectron Microscopy (IM)

For SEM, mature anthers and rice pollen grains were mounted on aluminum stubs by a piece of double-sided tape (Canemco, St. Laurent, Quebec, Canada) and were sputtered with gold. The specimens were observed in a Phillip 505 scanning electron microscopy (Philips Electron Optics, Eindhoven, The Netherlands).

For TEM, rice spikelets were fixed in 3% glutaraldehyde in 0.025 M phosphate buffer, pH 6.8 overnight at 1 hr 4° C. and postfixed in 1% osmium tetroxide on ice for 8 hrs. After dehydration in a graded ethanol series, the samples were embedded in acrylic resin (London Resin Company, Reading, Berkshire, UK). Ultra-thin sections (50-70 nm) were made using a Reichert Jung Ultracut E microtome (Leica, Vienna, Austria), and double-stained with 2% (w/v) uranyl acetate and 2.6% (w/v) lead citrate. The section was viewed and photographed with a Philips CM-10 transmission electron microscope (Philips Electron Optics).

For IM, developing anthers were fixed with 1.5% glutaraldehyde in 0.025 M phosphate buffer, pH 6.8 for 1 hr and then 3% glutaraldehyde in the same buffer for 3 hr at room temperature, rinsed with phosphate buffer at 4° C. overnight and dehydrated in a graded ethanol series. The fixed anthers were infiltrated with LR-white resin and polymerized with UV light. Sections (0.5 μm) were cut using a microtome (Reichert Ultracut E, BEMF, Honolulu, Hi., USA) and mounted on a silicone rubber plate (Canemco Inc, St. Laurent, Quebec, Canada) on a 300-mesh carbocoated nickel grid. The section was incubated with blocking solution containing 1% BSA in PBS buffer (10.14 mM Na₂HPO₄, 1.76 mM KH₂PO₄, pH 7.4, 136.9 mM NaCl, 2.69 mM KCl) for 30 min, followed with one hr incubation with the column purified antibody in the blocking solution (0.01 mg/ml). After washing 3 times with PBS buffer for 15 min, the section was reacted with the gold-labeled goat anti-rabbit IgG (EMGAR15; British BioCell International, Cardiff, UK) (100-fold dilution with the blocking solution) for 1 hr. The section was washed with 3 changes of PBS buffer for 15 min and 4 changes of distilled water for 12 min. The grid were stained with 2% uranyl acetate for 20 min, washed 4 times with distilled water for 16 min and incubated with 0.3% lead citrate for 10 min followed with 4 times of rinse with distilled water for 20 min. The section was viewed and photographed with a Philips 410 LS electron microscope (Philips Electron Optics). All the above steps were performed at RT except otherwise stated.

By way of examples, we have shown reduction of male fertility by silencing the expression of a RAFTIN gene in rice. It should be possible to enhance male fertility in plants by modulating appropriately sustained RAFTIN gene expression in a plant. For this purpose, a RAFTIN nucleotide sequence would be placed in sense orientation under the control of an anther-expressing promoter and using standard transformation vectors a plant would be transformed. The transformed cell is selected and grown into a plant and analyzed for male fertility at the time of flowering. The plant is found to have enhanced fertility.

### REFERENCES

Aarts, M. G. M., Hodge, R., Kalantidis, K., Florack, D., Wilson, Z. A., Mulligan, B., Stiekema, W. J., Scott, R. & Pereira, A. (1997). The Arabidopsis MALE STERILITY 2 protein shares similarity with reductases in elongation condensation complexes. Plant Journal 12, 615-623.
Bäumlein, H., Boerjan, W., Nagy, I., Bassüner, R., Van Montagu, M., D., I. & Wobus, U. (1991). A novel seed protein gene from Vicia faba is developmentally regulated in transgenic tobacco and Arabidopsis plants. Molecular General Genetics 225, 459-467.
Bedinger, P. A. (1992). The remarkable biology of pollen. Plant Cell 4, 879-887.
Chen, L., Miyazaki, C., Kojima, A., Saito, A. & Adachi, T. (1999). Isolation and characterization of a gene expressed during early embryo sac development in apomictic guinea grass (Panicum maximum). J. Plant Physiol. 154, 55-62.
Chen, L., Zhang, S., Beachy, R. N. & Fauquet, C. M. (1998). A protocol for consistent, large-scale production of fertile transgenic rice plants. Plant Cell Report 18: 25-31. Plant Cell Report 18, 25-31.
Clough, S. J. & Bent, A. F. (1998). Floral dip: a simplified method for Agrobacterium-mediated transformation of Arabidopsis thaliana. The Plant Journal 16, 735-743.
D'Arcy, W. G. (1996). Anthers and stamens and what they do. In The anther: form, function and phylogeny, pp. 1-24. Edited by W. G. D'Arcy & R. C. Keating. Cambridge, UK: Cambridge University Press.
Datla, P, S. S., Hammerlindl, J. K., Panchuk, B., Pelcher, L. E. & Keller, W. (1992). Modified binary plant transformation vectors with the wild-type gene encoding NPTII. Gene 122, 383-384.
Datta, N., LaFayette, P. R., Kroner, P. A., Nagao, R. T. & HKey, J. L. (1993). Isolation and charcaterization of three families of auxin down-regulated cDNA clones. Plant Molecular Biology 21, 859-869.
De Block, M., Debrouwer, D. & Moens, T. (1997). The development of a nuclear male sterility system in wheat. Expression of the barnase gene under the control of tapetum specific promoters. Theor Appl Genet 95, 125-131.
Dorion, S., Lalonde, S. & Saini, H. S. (1996). Induction of male sterility in wheat (Triticum aestivum L.) by meiotic-stage water deficit is preceded by a decline in invertase activity and changes in carbohydrate metabolism in anthers. Plant Physiology 111, 137-145.
El-Ghazaly, G. & Jensen, W. A. (1986). Studies of the development of wheat (Triticum aestivum) pollen I. Formation of the pollen wall and Ubisch bodies. Grana 25, 1-29.
El-Ghazaly, G. & Jensen, W. A. (1987). Development of wheat (Triticum aestivum) pollen. II Histochemical differentiation of wall and Ubisch bodies during development. American Journal of Botany 74, 1396-1418.
Furness, C. A. & Rudall, P. J. (2001). The tapetum in basal angiosperms: early diversity. International Journal of Plant Sciences 162, 375-392.
Goff, S. A. et al. (2002). A draft sequence of the rice genome (Oryza sativa L. ssp. japonica). Science 296, 92-100.
Goldberg, R. B., Beals, T. P. & Sanders, P. M. (1993). Anther development: basic principles and practical applications. Plant Cell 5, 1217-1229.
Hattori, J., Boutilier, K A., van Lookeren Campagne, M. M. & Miki, B. L. (1998). A conserved BURP domain defines a novel group of plant proteins with unusual primary structures. Molecular General Genetics 259,424-428.
Huang, J., Hirji, R., Adam, L., Rozwadowski, K. L., Hammerlindl, J. K, Keller, W. A. & Selvaraj, G. (2000). Genetic engineering of glycinebetaine production toward enhancing stress tolerance in plants: metabolic limitations. Plant Physiology 122, 747-756.
Huysmans, S., El-Ghazaly, G. & Smets, E. (1998). Orbicules in angiosperm: morphology, function, distribution, and relation with tapetum types. Bot. Rev. 64, 240-272.
Koltunow, A. M., Truettner, J., Cox, K. H., Wallroth, M. & Goldberg, R. B. (1990). Different temporal and spatial gene expression patterns occur during anther development. Plant Cell 2, 1201-1224.
Laemmli, U. K. (1970). Cleavage of structural proteins during assembly of the head of bacteriophage T4. Nature (London) 227, 680-685.
Lalonde, S., Beebe, D. U. & Saini, S. (1997). Early signs of disruption of wheat anther development associated with the induction of male sterility by meiotic-stage water deficit. Sexual Plant Reproduction 10, 40-48.
Mariani, C., Gossele, V., de Beuckeleer, M., de Block, M., Goldberg, R. B., de Greef, W. & Leemans, J. (1992). A chimaeric ribonuclease-inhibitor gene restores fertility to male sterile plants. Nature 357, 384-387.
Mascarenhas, J. P. (1975). The biochemistry of angiosperm pollen development. Bot Rev 41, 259-314.
Mascarenhas, J. P. (1990). Gene activity during pollen development. Ann. Rev. Plant Physiol. Plant Mol. Biol. 41, 317-338.
McCormick, S. (1993). Male gametophyte development. Plant Cell 5, 1266-1275.
Mizelle, M. B., Sethi, R, Ashton, M. E. & Jensen, W. A. (1989). Development of the pollen grain and tapetum of wheat (Triticum aestivum) in untreated plants and plants treated with chemical hybridizing agent RH00007. Sex Plan Reprod 2, 231-253.
Piffanelli, P., Rogers, J. H. E. & Murphy, D. J. (1997). Intra and extracellular lipid composition and associated gene expression patterns during pollen development in Brassica napus. Plant Journal 11: 549-652.
Ragland, M. & Soliman, K. M. (1997). Sali5-4a and Sali3-2, two genes induced by aluminum in soybean roots. Plant Physiology 114, 395.
Sanders, P. M., Bui, A. Q., Weterings, K., McIntire, K. N., Hsu, Y.-C., Lee, P. Y., Truong, M. T., Beals, T. P. & Goldberg, R. B. (1999). Anther developmental defects in Arabidopsis thaliana male-sterile mutants. Sex Plan Reprod 11, 297-322.
Shivanna, K. R., Cresti, M. & Ciampolini, F. (1997). Pollen development and pollen-pistil interaction. In Pollen biotechnology for crop production and improvement. Edited by K. R. Shivanna & V. K. Sawhney. Cambridge, United Kingdom: Cambridge University Press.
Smith, N. A., Singh, S. P., Wang, M.-B., Stoutjesdijk, P. A., Green, A. G. & Waterhouse, P. M. (2000). Total silencing by intron-spliced hairpin RNAs. Nature 407, 319-320.
Suarez-Cervera, M., Marquez, J. & Seoane-Camba, J. (1995). Pollen grain and Ubisch body development in Platanus acerifolia. Rev. Palaeobot. Palynol 85, 63-84.
The Arabidopsis Genome Initiative (2000). Analysis of the genome sequence of the flowering plant Arabidopsis thaliana. Nature 408, 796-815.
Wan, L., xia, Q., Qiu, X. & Selvaraj, G. (2002). Early stages of seed development in Brassica napus: a seed coa-specific cysteine proteinase associated with programmed cell death of the inner integument. Plant Journal 30, 1-10.
Wang, A., Carrier, K., Chisholm, J., Wieczorek, A., Huguenot, C. & Sanfacon, H. (1999). Porteolytic processing of tomato ringspot nepovirus 3C-like protease precursors: definition of the domains for the VPg, protease and putative RNA-dependent RNA polymerase. Journal of General Virology 80, 799-809.
Wang, A. & Sanfacon, H. (2000). Proteolytic processing at a novel cleavage site in the N-terminal region of the tomato ringspot nepovinms RNA-1-encoded polyprotein in vitro. Journal of General Virology 81, 2771-2781.
Wang, A., Xia, Q., Xie, W., Dumonceaux, T., Zou, J., Datla, R. & Selvaraj, G. (2002). Male gametophyte development in bread wheat (Triticum aestivum L.): molecular, cellular, and biochemical analyses of a sporophytic contribution to pollen wall ontogeny. Plant Journal 30, 613-623.
Wiermann, R. & Gubatz, S. (1992). Pollen wall and sporopollenin. Int. Rev. Cytol. 140, 35-72.
Yamaguchi-Shinozaki, K. & Shinozaki, K. (1993). The Plant hormone abscisic acid mediates the drought-induced expression but not the seed-specific expression of rd22, a gene responsive to dehydration stress in Arabidopsis thaliana. Molecular General Genetics 238, 17-25.
Yu, J. et al. (2002). A draft sequence of the rice genome (Oryza sativa L. ssp. indica). Science 296, 79-92.
Zhang, Y., Shewry, P. R., Jones, H., Barcelo, P., Lazzeri, P. A. & Halford, N. G. (2001). Expression of antisense SnRK1 protein kinase sequence causes abnormal pollen development and male sterility in transgenic barley. Plant Journal 28, 431-441.
Zheng, L., Heupel, R. C. & DellPenna, D. (1992). The β subunit of tomato fruit polygalacturonase isoenzyme 1: isolation, characterization, and identification of unique structural features. Plant Cell 4, 1147-1156.

1. An isolated RAFTIN nucleotide sequence.

2. The isolated RAFTIN nucleotide sequence of claim 1 comprising a BURP domain of two conserved phenylalanine residues (FF) at the N-terminus of the domain and a consensus sequence of the formula CHX₁₀CHX₂₅₋₂₇CHX₂₄₋₂₅CH wherein CH represents each of four repeated cysteine-histidine (CH) motifs in the consensus sequence and X represents any amino acid.

3. The isolated RAFTIN nucleotide sequence of claim 1 in the form of genomic DNA.

4. The isolated RAFTIN nucleotide sequence of claim 1 in the form of cDNA.

5. The isolated RAFTIN nucleotide sequence according to claim 1 comprising a RAFTIN gene promoter sequence.

6. The isolated RAFTIN nucleotide sequence according to claim 1 comprising a RAFTIN protein encoding sequence.

7. A transformation vector comprising a RAFTIN nucleotide sequence according to claim 1.

8. A plant cell comprising the transformation vector of claim 7.

9. A plant comprising the transformation vector of claim 7.

10. A seed of a plant comprising the transformation vector of claim 7.

11. An RNA hairpin construct comprising a promoter operably linked to sense-oriented RAFTIN nucleotide sequence, an intron and an antisense-oriented RAFTIN nucleotide sequence.

12. A transformation vector comprising a sequence encoding the RNA hairpin construct of claim 11.

13. The transformation vector of claim 12 wherein said sequence encoding the RNA hairpin construct is operably linked to and under the control of a RAFTIN gene promoter sequence.

14. A method for producing a cell of a plant having male sterility or modulated male fertility which method comprises transforming said cell with the transformation vector of claim 12.

15. A method for producing a plant having male sterility or modulated male fertility which method comprises transforming a cell of said plant with the transformation vector of claim 12 and multiplying said cell to yield said plant.

16. A transformed plant cell comprising a sequence encoding the RNA hairpin construct of claim 11.

17. A male-sterile plant or seed thereof comprising transformed plant cells as defined in claim 16.

18. A method for producing a cell of a plant having enhanced male fertility which method comprises transforming said cell with the transformation vector of claim 7.

19. A method for producing a plant having enhanced male fertility which method comprises transforming said cell with the transformation vector of claim 7 and multiplying said cell to produce said plant.


SEQUENCE LISTING (43 sequences)

SEQ ID NO: 1 - DNA, 17 bp, Artificial
  Primer OL3044
tgccacactcgccattg

SEQ ID NO: 2 - DNA, 17 bp, artificial
  Primer OL3045
tttccagcgaggctgct

SEQ ID NO: 3 - DNA, 29 bp, artificial
  Primer OL4382
cggggtaccgaacgcttccatggcgcgct

SEQ ID NO: 4 - DNA, 29 bp, artificial
  Primer OL4383
gctctagagcttctacgcccgtcgagctc

SEQ ID NO: 5 - DNA, 18 bp, artificial
  Primer OL3073
ctccatgtccaccatgta

SEQ ID NO: 6 - DNA, 18 bp, artificial
  Primer OL3148
cgacgtatttgtcgtagt

SEQ ID NO: 7 - DNA, 17 bp, artificial
  Primer OL3815
tctcgaacgcttccatg

SEQ ID NO: 8 - DNA, 18 bp, artificial
  Primer OL4556
tcgagctcgtcgccgtca

SEQ ID NO: 9 - DNA, 18 bp, artificial
  Primer OL4557
gcagcaccagtgctgctg

SEQ ID NO: 10 - DNA, 23 bp, artificial
  Primer
cgggatccgcgcttcctcgtcgc

SEQ ID NO: 11 - DNA, 25 bp, artificial
  Primer
ggaattctcacgccggcgagcgatt

SEQ ID NO: 12 - DNA, 27 bp, artificial
  Primer OL3070
tccagcctgaaccgcgaccagggtggt

SEQ ID NO: 13 - DNA, 26 bp, artificial
  Primer 3071
gtggtggcgaggagggcgacgaggaa

SEQ ID NO: 14 - DNA, 14 bp, artificial
  Primer 3079
cgaaggactctggt

SEQ ID NO: 15 - DNA, 17 bp, artificial
  Primer 3080
catggaagcgttcgaga

SEQ ID NO: 16 - DNA, 27 bp, artificial
  Primer 3861
cccaagcttctgtcgatggcgctctgt

SEQ ID NO: 17 - DNA, 26 bp, artificial
  Primer 3862
cgggatccgatgtgcgctaggtgaga

SEQ ID NO: 18 - DNA, 30 bp, artificial
  Primer 3863
caagcttctagacttgttgagtgccacact

SEQ ID NO: 19 - DNA, 25 bp, artificial
  Primer 3142
cccaagctttacccacacgtcatga

SEQ ID NO: 20 - DNA, 25 bp, artificial
  Primer 3143
cgggatcccatggaagcgttcgaga

SEQ ID NO: 21 - DNA, 25 bp, artificial
  Primer 3888
cgggatccgacgtatttgtcgtagt

SEQ ID NO: 22 - DNA, 25 bp, artificial
  Primer 3889
ggactagtcagcttcgtcgtcggca

SEQ ID NO: 23 - DNA, 23 bp, artificial
  Primer 3886
gctctagacgccttcctccgcct

SEQ ID NO: 24 - DNA, 24 bp, artificial
  Primer 3887
ggaattcgacgtatttgtcgtagt

SEQ ID NO: 25 - DNA, 25 bp, artificial
  Primer 3892
cgggatcctgggagcctcttgccga

SEQ ID NO: 26 - DNA, 25 bp, artificial
  Primer 3893
ggactagtcacagaagccaccagct

SEQ ID NO: 27 - DNA, 23 bp, artificial
  Primer 3890
gctctagacgccgttctccgcct

SEQ ID NO: 28 - DNA, 24 bp, artificial
  primer 3891
ggaattctgggagcctgttgccga

SEQ ID NO: 29 - DNA, 1356 bp, Triticum aestivum
ctctggacctctcacctagcgcacatccatggcgcgcttcctcgtcgccctcctcgccaccaccctggtcgcggttcaggctggagggcagctgggccacgcggcgccggcgacggcggaggtgttctggcgcgccgtgctgccacactcgccattgcccgacgccgttctccgccttctcaaacaacccgcagcaggtgttgaactgctcacagaagccaccagcttcgtgagggatgccgaggacaggccccccttcgactaccgtgattacagccgctcgccgcccgatgatgaaccgagcaagagcaccggcgccgcctccggggcgcgggacttcgactacgacgactacagcgggggcgacaagctccgtggcgccgcctccggggcgcgggacttcgactacgacgactacagcggggccgacaagctccgtggcgccaccgatgaatacaaggcgccgagcagcagcctcgctggaaacggggcgtccatggctaggggcggcaaggcggagacgacgacggtgttctttcacgaggaggcggtgcgcgtcggcaagaggctcccattccgcttcccgccggcgactcccgccgcgctcggtttcctgccgcgccaggtcgccgactccgtcccgttcacgacggccgcgctgcctggcgtcctcgcgacgttcggcgtcgcgtccgactccgccacggtggccagcatggaggcgacgctgcgcgcctgcgagtcgccgaccatcgccggggagtccaagttctgcgcgacctcgctggaggccctggtggagcgcgccatggaagtgctggggacccgcgacatcaggccggtgacgtcgacgctgccccgcgccggcgccccgctgcagacgtacaccgtccgctccgtgcggccggtggagggggggcctgtcttcgtggcgtgccacgacgaggcctacccgtacaccgtgtaccggtgccacaccactggcccgtccagggcgtacatggtggacatggagggcgcgcgcggcggcgacgcggtgaccatcgccaccgtgtgccacaccgacacgtccctgtggaacccggagcacgtctccttcaagctcctgggcaccaagcctggcggcacgccggtctgccacctcatgccgtacgggcacataatctgggccaagaacgtgaatcgctcgccggcgtgagcggcccgggcagctctgtggtctcgccggaactaagatcgatgtactactactactatctgtttccacctacgtcttctgttgttcagaccaccagatggtcaccagagcagcgcttgtaataaaagaacagcttctgcaaaaaaaaaaaaaaaaaa

SEQ ID NO: 30 - DNA, 1560 bp, Triticum aestivum
ctctggacctctcacctagcgcacatccatggcgcgcttcctcgtcgccctcctcgccaccaccctggtcgcggtaatggccgaagaagccactgagcaacgcctgcatcttcttcatttcggcaaactgcacctagtgcatttcgcatgagattgatcgatcacaaactggtgctaacggcctgtttcgtcacaggttcaggctggagggcagctgggccacgcggcgccggcgacggcggaggtgttctggcgcgccgtgctgccacactcgccattgcccgacgccgttctccgccttctcaaacaacccgcagcaggtctgtctttcatgttcctttcctcgtcgccctccgttaactgtcttcttctctcgagtttgattgaccgccaaacacaaaaaatgcatgcacgcacaggtgttgaactgctcacagaagccaccagcttcgtgagggatgccgaggacaggccccccttcgactaccgtgattacagccgctcgccgcccgatgatgaaccgagcaagagcaccggcgccgcctccggggcgcgggacttcgactacgacgactacagcgggggcgacaagctccgtggcgccgcctccggggcgcgggacttcgactacgacgactacagcggggccgacaagctccgtggcgccaccgatgaatacaaggcgccgagcagcagcctcgctggaaacggggcgtccatggctaggggcggcaaggcggagacgacgacggtgttctttcacgaggaggcggtgcgcgtcggcaagaggctcccattccgcttcccgccggcgactcccgccgcgctcggtttcctgccgcgccaggtcgccgactccgtcccgttcacgacggccgcgctgcctggcgtcctcgcgacgttcggcgtcgcgtccgactccgccacggtggccagcatggaggcgacgctgcgcgcctgcgagtcgccgaccatcgccggggagtccaagttctgcgcgacctcgctggaggccctggtggagcgcgccatggaagtgctggggacccgcgacatcaggccggtgacgtcgacgctgccccgcgccggcgccccgctgcagacgtacaccgtccgctccgtgcggccggtggagggggggcctgtcttcgtggcgtgccacgacgaggcctacccgtacaccgtgtaccggtgccacaccactggcccgtccagggcgtacatggtggacatggagggcgcgcgcggcggcgacgcggtgaccatcgccaccgtgtgccacaccgacacgtccctgtggaacccggagcacgtctccttcaagctcctgggcaccaagcctggcggcacgccggtctgccacctcatgccgtacgggcacataatctgggccaagaacgtgaatcgctcgccggcgtgagcggcccgggcagctctgtggtctcgccggaactaagatcgatgtactactactactatctgtttccacctacgtcttctgttgttcagaccaccagatggtcaccagagcagcgcttgtaataaaagaacagcttctgc

SEQ ID NO: 31 - DNA, 1719 bp, Triticum aestivum
ctgtcgatggcgctctgtccttgtgattctttcttagggaactcgtctctggggcctccgaggcctgcaaccctgtatcaggacaattctgactggcctccaggagtcctaacagccaccgacctggtccactgggcccatctagagtatcttgaagtgtcgtttgcacaaatcccgctaattaagggatgtgatgatgatggtttctgaatccgcgcgccttacctcgcaaaacggggaattgcaaaggatatatggcacctgtcgcgtcgtgaggccagacgcttcggtttcaagctggttatagggagggggaaacgaagggttttttctccctctgtcttcatccattttcgtctcccagccctcagctcccaaaagcgtgtcgccacctcaaagtcttcagcgcttgctcacgtagcccccgtccaccccttccttgccaccaagatggcccgaaccaagagcgagaaggttcctaaggttcccagctaggatctgcccgccgctggaacggggctgaagcggaagagggtcgcctccaagggtggtatgaaacaacagccggaagcccccaagactacaggaaagtggttcccttcctcggccaccgacaaaaaacttcagggtctcgtggagatagggctgatgccagcggatttggagtgccgcctcccgggggacgaggctccgccaactcctcgcgacggtgagcacatcctctgcctggagtatatatttcgggaggggctcgggtttcccctacacgacttcgtttgcgggatcttgcgcttctacggctgctagctacaccacatcccgtcaaacggggttctttacattgcaaacttcatcacattttgcgagtgctttctcgggactgccgctcactttaagttgttccaatacttcaatcaggactgcgttcagaccaacggggacatcgtctacgaccccgcaacaccaaattcctcgccacatacctccggaaaataatcctatacaacctggtctcacgcttcatctcgtaagatttgccatgtgtacttcaccaatcttgatgcatccctttttccccaagatttatatgcctgatctgtattttgtctccgctgtttcgagatttgatgtttaattgatgaagcccaagcaatccggcatgcccgtcggtgcactagatggctagcttttctacggtgctgggcctgccggcgaggggcgcgaggccacgtaggagactgttaggattcatggggctggacgctggtggcgtgaagttcgggaaggaggattgaggaagaaggatgcatcaagattggtgaagaacacgtggcatcctctagagtaggtcttacgagatgaagcctgagaccaggtcgtatgggattattttcccggacctcccgaagccccgcaaagttaactgcagctgcgtggacggcgagcaccgcaccgcacacgaacgcgaacctgacgctgccgcgccacacaacacgccattcgcgcgcggatcgtcggatgtcacgcccaggattatattctccggtgccgcacgtaccatgcgatcgcacagctcacgtcgagagcttttctgtttggcgtcgccgtcaatgaaacaccttcccgtcgagccgacgacgcctataagtacctcgtctgatcgcatcatcactcccaagtactacaacctctggacctctcacctagcgcacatccatg

SEQ ID NO: 32 - DNA, 1285 bp, Triticum aestivum
cgacctctcacctagcgcacatccatggcgcgcttcctcgtcgccctcctcgctgccaccctggtcgcggttcaggctggagggcagctgggccacgcggcgccggctacgggggaggtgttctggcgcgccgtgctgccgcactcgccattgcctgacgccgttctccgcctcctcaaacaacctgcagcagaatccaccagcttcgtgagagaccccgaggacaggccccccttcgactaccgtgattacagccgctcgtcgtccgatgatgaaccgagcaagagcaccgtcgccgcctccggagcggggggcttcgactacgacaactacagcggggccgacgaacgtcgtggtgccaccgatgaatacaaggcgccgagcagcagcctcgctggaagcggggcgtacatggctaggggcggcaaggcggagacgacgacggtgttctttcacgaggaggcggtgcgcgtcggcaggaggctcccattccacttcccgccggcgactcccgccgctctcggtttcctgccgcgccaggtcgccgactccgtcccgttcacgacggccgcgctgcccggcatcctcgcgacgtttggcatcgcgtccgactccaccacggtgcccagcatggaggcgacgctgcgcgcctgcgagtcgcccaccatcgccggggagtccaagttctgcgcgacttcgctggaggccctggtggagcgcgccatgggagtgctggggacccgggacatcaggccggtgacgtcgacgctgccccgcgccggcgccccgctgcagacgtacaccgtcgtcgccgtgcagccggtggagggggggcctgtcttcgtggcgtgccacgacgaggcctacccgtacaccgtgtaccggtgccacaccaccggcccgtccagggcgtacacggtggacatggagggcgcgcgcggcgccgacgcggtgaccatcgccgccgtgtgccacaccgacacgtccctgtggaacccggagcacgtctccttcaagctcctcggcaccaagcccggcggcacgccggtctgccacctcatgccgtacgggcacataatctgggccaagaacgtgaagcgctcgccggcgtgagcggccttgcagctctgtggtgtcgccggaactaagatcgatgtactactactatctgttcctacctacgtcttcttgttgttcataccaccagatggtcacccaagagcaagcgttcgtaataaaaagaacagctttttgcagaagctggtgttttattttaaaaaaaaaa

SEQ ID NO: 33 - DNA, 1503 bp, Triticum aestivum
cgacctctcacctagcgcacatccatggcgcgcttcctcgtcgccctcctcgctgccaccctggtcgcggtaatggccgaagaagagcaacgcctgcatcttcttcattttggcaaattgcacctagtacattttgcatgagattaatcaatcacaaactggtgctaacggcctgtttcgtcccaggttcaggctggagggcagctgggccacgcggcgccggctacgggggaggtgttctggcgcgccgtgctgccgcactcgccattgcctgacgccgttctccgcctcctcaaacaacctgcagcaggtctgtcttgcatcttcctcgtcgccctccgttaactgtcttcttctctcgagtttgattgatcaccaaacacaaaaatgcatgcacgcgtgggtgttgaactgcgcacagaatccaccagcttcgtgagagaccccgaggacaggccccccttcgactaccgtgattacagccgctcgtcgtccgatgatgaaccgagcaagagcaccgtcgccgcctccggagcggggggcttcgactacgacaactacagcggggccgacgaacgtcgtggtgccaccgatgaatacaaggcgccgagcagcagcctcgctggaagcggggcgtacatggctaggggcggcaaggcggagacgacgacggtgttctttcacgaggaggcggtgcgcgtcggcaggaggctcccattccacttcccgccggcgactcccgccgctctcggtttcctgccgcgccaggtcgccgactccgtcccgttcacgacggccgcgctgcccggcatcctcgcgacgtttggcatcgcgtccgactccaccacggtgcccagcatggaggcgacgctgcgcgcctgcgagtcgcccaccatcgccggggagtccaagttctgcgcgacttcgctggaggccctggtggagcgcgccatgggagtgctggggacccgggacatcaggccggtgacgtcgacgctgccccgcgccggcgccccgctgcagacgtacaccgtcgtcgccgtgcagccggtggagggggggcctgtcttcgtggcgtgccacgacgaggcctacccgtacaccgtgtaccggtgccacaccaccggcccgtccagggcgtacacggtggacatggagggcgcgcgcggcgccgacgcggtgaccatcgccgccgtgtgccacaccgacacgtccctgtggaacccggagcacgtctccttcaagctcctcggcaccaagcccggcggcacgccggtctgccacctcatgccgtacgggcacataatctgggccaagaacgtgaagcgctcgccggcgtgagcggccttgcagctctgtggtgtcgccggaactaagatcgatgtactactactatctgttcctacctacgtcttcttgttgttcataccaccagatggtcacccaagagcaagcgttcgtaataaaaagaacagctttttgcagaagctggtgttttatttt

SEQ ID NO: 34 - DNA, 2095 bp, Triticum aestivum
ttgttgagtgccacactatattcactacaccatatgcacattatgcttggattgtcttgtacttgactcatgtgtttagacacttcattttatttggtgttgtgaatgactcctatgcttaccatagacctttcattgagcgctttgtgcatgtttgttataccttgaggtagatgtttgttctcttgtcaaatatatagcatctctacctcccatttgcatgcttgtttccatgatgtccttgattgtgctcaattcatatgcttctgtgacatgccacaatcctttgtcacaccatatgctaggcttgatgatgacacttgttgggtgactcaccttttgaatgattggttttgcattaacgctaaccacatttatttttccaagtgtttgttgtccttgctccttttgaaggaaccacatgacggtgcgacattggagagtgcctatttcgagcttcaagatgatgagtgcttggtgatcgtccacttctacatggtgacgccgtctctttcccatggtgatttggtttttgatccgaggtcggatctttcccaagtgggaggggatgatgcggagcatactacggacatcaccatgtctagagttcattcagcaagtgacacctatcacatctacttcacatacataaaggtgaatcatctcctttacacgtgctcacttgatcccttcgaggatggtatactacttgacacttctcacgtgtgcatgcataggcattgtcggagcaccatgaacgatgaggaggagtgcgagcacaagtgtacaactacaccatccgcgagggaagcatggaagagaaggaagaagaagcatggacaagcttctggaaagcccggaacttctggcctcctgcccggaacttccggtcatccgaaacttcctgccccgacacaccgaagccgtctgagagcgtgccaaatctctggatagcccggaccttcgaccggaacctccggcgcctggaccttccggccatccctggaactcccggcctgcctgcacgcagagactcgggccgaagcgcatgtaccctttcgcccctcacttatcccttcgtggctatcactatatatactcatcctcctcctccattctagggttagcattttgatagctcatttgcatgtgagatttgctccttacccccatctcctcttgagagagtgagattgatgcactccattggagtccaaggtctcctttggagaagatcccataggggaatcaagaccccatcatgggaagatccttctaggattcaagacctcaactcctttaaggattgggatgaactagttacctcttgtatcttcttgtgttggatttaaacctttgtatccctctatgtgtatgtggatttagcatatgtgtgattggatcttgtctattggagtgtttcctctcttttgttttccttgtgttcatcgttttcttcgggagatcccctccatttcgtgaaagatcggtccctagggttctaccctacattagctcaggtttcccctacacatcttcgtttgtgagctgttgcgcttctacggctgggagctacagcacatctcattcccaccaaacggggttcttcacattgtaaacttcatcgtattttgcgaatgctttctggggacagccactcactttgagttgttccgatacttcttccgggtctgcgttcagaccaacggggacaccgtctgcaaccttggaggagccattcctgcgacacaccaaaattttcgccacggaccccccgaagatccgcaagaaaaaaaaagctgcaacggcgtggacggcgagcaccgcaccgcacacgaacgcgaacgcgacgctgccgcgccacacaacacgccattcgcgcgcggatcgtcggatgtcacgcccacgataatattctccggtgccgcacgtaccatgcgatcgcacagctcacatcgagagcttttctgtttggtgtcgccgtcaatgaaacaccttcccgtcaagccgacgacgcctataagtacctcgcctgatcgcattatcactcccaagtactacaacctctcgacctctcacctagcgcacatccatg

SEQ ID NO: 35 - DNA, 246 bp, Triticum aestivum
atggcgcgcttcctcgtcgccctcctcgctgccaccctggtcgcggttcaggctggagggcagctgggccacgcagcgccggcgacggcggaggtgttctggcgcgccgtgctgccgcactcgccattgcccgacgccgttctccgcctcctcaaacaacctgcagcaggtgttgaactgcacacagaagccaccagcttcgtaagagaccccgaggacaggccccccttcgactaccgtgattac

SEQ ID NO: 36 - DNA, 441 bp, Triticum aestivum
atggcgcgcttcctcgtcgccctcctcgctgccaccctggtcgcggtaatggccgaagaagccactgagcaacgcctgcatcttctttattttggcaaactggtgctaacggccaatactgccgcttgcgttacgtctcaggttcaggctggagggcagctgggccacgcagcgccggcgacggcggaggtgttctggcgcgccgtgctgccgcactcgccattgcccgacgccgttctccgcctcctcaaacaacctgcagcaggtctgtcttgcatgttcctcgtcgccctccgttaactgtcttcttctctcgagtttgattgatcaccaaacacaaaaatgcatgcacgcgtacgcgtaggtgttgaactgcacacagaagccaccagcttcgtaagagaccccgaggacaggccccccttcgactaccgtgattac

SEQ ID NO: 37 - DNA, 1301 bp, Oryza sativa
gtcgcagtcgtctccggcgagaaatcggctgcgccccgtctctctctctctcgaacgcttccatggcgcgcttcctcctcctcctcgtcgccgtcgccgctgccgccgccgtgctttcgctgggcgacgcggcgccgtcgacggccgaggtgttctggcgcgccgtgctgccggaatccccgttgccggacgccttcctccgcctcctccgccctgacaccagcttcgtcgtcggcaaagcggaggcggccggtggcgcggcgcggaccggattccccttcgattacactgactacaggggatctgattctccgacgacggcgagtggtttggacctcgccggtgacttcggcgagccggcgcctttcggctacgactacagtgcacagggcgaaggcggcggcggcggcgccgccgccgccgcgggagagcaggttcttgccgtcgacgcgggcttcaactacgacaaatacgtcggcgcgaggaagctccgcggcggcagcagcaccgccggcggagagaatgatgacgagcctttcgggtacgactacaaggcgccgagcagcggcagcggcaccgcggcgtcgacgacggcgcgaggcgtcggcacgggcgccacgacgacggtgttcttccacgaggaggcggtgcgcgtcggcgagaggctcccgttctacttcccggcggcgacgacgtcggcgctgggcttcctgccgcgccgcgtcgcggactccatcccgttcacggcggccgcgctgccggccgtcctcgcgctgttcggcgtcgcgccggacaccgccgaggcggccggcatgagggagacgctgcgcacgtgcgagtggccgaccctcgccggcgagtccaagttctgcgccacgtcgctggaggccctggtggagggcgccatggcggcgctcgggacacgcgacatcgccgcgctggcgtcgacgctgccccgcggcggcgcgccgctgcaggcgtacgccgtccgcgccgtgctccccgtcgagggcgccggcttcgtggcgtgccacgaccaggcgtacccgtacaccgtgtaccgctgccacaccaccggcccggccagagcttacatggtggagatggaaggcgacggcggcggcgatggcggcgaggcggtgaccgtggccaccgtgtgccacaccaacacgtcgcggtggaacccggagcacgtctcgttcaagctcctcggcaccaagcccggcggctcgccggtgtgccacctcatgccgtacgggcacatcgtctgggccaagaacgtgaagagctcgacggcgtag

SEQ ID NO: 38 - DNA, 1479 bp, Oryza sativa
gtcgcagtcgtctccggcgagaaatcggctgcgccccgtctctctctctctcgaacgcttccatggcgcgcttcctcctcctcctcgtcgccgtcgccgctgccgccgccgtgctttcggtacactcatgatgccgctactcagctgagccatgcaccgttgcacccgtatactaacgatcgctcgatcgaccgacgatgtgtgttcttcagcagctgggcgacgcggcgccgtcgacggccgaggtgttctggcgcgccgtgctgccggaatccccgttgccggacgccttcctccgcctcctccgccctggtcggtgtccttccttcctccttccgccgccgcgcgccgccattactctcctcgaggtttgatttgtttgtggacgttgcagacaccagcttcgtcgtcggcaaagcggaggcggccggtggcgcggcgcggaccggattccccttcgattacactgactacaggggatctgattctccgacgacggcgagtggtttggacctcgccggtgacttcggcgagccggcgcctttcggctacgactacagtgcacagggcgaaggcggcggcggcggcgccgccgccgccgcgggagagcaggttcttgccgtcgacgcgggcttcaactacgacaaatacgtcggcgcgaggaagctccgcggcggcagcagcaccgccggcggagagaatgatgacgagcctttcgggtacgactacaaggcgccgagcagcggcagcggcaccgcggcgtcgacgacggcgcgaggcgtcggcacgggcgccacgacgacggtgttcttccacgaggaggcggtgcgcgtcggcgagaggctcccgttctacttcccggcggcgacgacgtcggcgctgggcttcctgccgcgccgcgtcgcggactccatcccgttcacggcggccgcgctgccggccgtcctcgcgctgttcggcgtcgcgccggacaccgccgaggcggccggcatgagggagacgctgcgcacgtgcgagtggccgaccctcgccggcgagtccaagttctgcgccacgtcgctggaggccctggtggagggcgccatggcggcgctcgggacacgcgacatcgccgcgctggcgtcgacgctgccccgcggcggcgcgccgctgcaggcgtacgccgtccgcgccgtgctccccgtcgagggcgccggcttcgtggcgtgccacgaccaggcgtacccgtacaccgtgtaccgctgccacaccaccggcccggccagagcttacatggtggagatggaaggcgacggcggcggcgatggcggcgaggcggtgaccgtggccaccgtgtgccacaccaacacgtcgcggtggaacccggagcacgtctcgttcaagctcctcggcaccaagcccggcggctcgccggtgtgccacctcatgccgtacgggcacatcgtctgggccaagaacgtgaagagctcgacggcgtag

SEQ ID NO: 39 - DNA, 1461 bp, Oryza sativa
cgaaggcaaactctggtaaggattcccattacacgaatcaatttaataagtctaaaacgaacactatgttatgagaaacacctcacatccgtccataaccgtgggcatgactatttaaaaagtttaactaaactctacaaaagttgcacgctttacccacacgtcatgaacgtttcacattaccgaatacatgtggatcggacatggccgacaaaggagagttcaatacaaggcttttccataaccaatccataaatatcctatgtcccacggttgggtggaatctctccaccaaacatcaagccaggatcaggtcctcatctacccatgccccactccatggactccgacacatccccactgcaggagattgccatatacgccaccataccagtgctcctcaaccgctaacatgttggacaccaaattctatatacttatatagttcatctccactaagtgtagttaattacatttctctcttctctcattaagccacatcacctcaattatttttagcctttagatgatagatctatggtccaaattgtcttttctttcttctctcttaaaaacatgcaatcttaaatacttttaggctcaaaattgtatcaaattgttttagttttgtacatattatgcaacttaatttttcgccgcaacgcggaggggtatttcatctagtattatttaagagctatacacactgctataggggaaaaaaaagataggtttggccccctggtcagtcctgttgcacggctatatgttgaagggaaaaagccagtacgttttgtaggttgttttttttttagaattgctaaaaagttgtggcatgttttttaggtaaaagcctttaaatataagttacattgtaactacagtgtaattccgctgtaactatattgtaatctctatataagttagatataaaattacatatatattattttaatacttatttataagttagtatattatagttataatggaattaattataattatagtatagttagatttgaaagtttttcctttaagaaatttcgcaacagtttattagatatagtccctaaacgaaaatgtcaggtggatgcatgattcagtgtgacgctcgggcggatcacggctgcgtcacgaaaattccccccatgcaacccgcgtccggccgtccttcgtgccaacaggcaacagcgcggcgccggcgaacgtcacgcccaagattatattccccctctcgcgctcgcgcgcgccgcgacgtcgtcggagccaacattatttttctgtttcctgtcaccgtcgccgttgatctcaagcgagatttgaggtttggccacgacgacgcctgcctataaataccaggtggtggtcaccgcccggcggcgtcgatcgatccgtcgcagtcgtctccggcgagaaatcggctgcgccccgtctctctctctctcgaacgcttccatg

SEQ ID NO: 40 - protein, 389 aa, Triticum aestivum
MARFLVALLATTLVAVQAGGQLGHAAPATAEVFWRAVLPHSPLPDAVLRLLKQPAAGVELLTEATSFVRDAEDRPPFDYRDYSRSPPDDEPSKSTGAASGARDFDYDDYSGGDKLRGAASGARDFDYDDYSGADKLRGATDEYKAPSSSLAGNGASMARGGKAETTTVFFHEEAVRVGKRLPFRFPPATPAALGFLPRQVADSVPFTTAALPGVLATFGVASDSATVASMEATLRACESPTIAGESKFCATSLEALVERAMEVLGTRDIRPVTSTLPRAGAPLQTYTVRSVRPVEGGPVFVACHDEAYPYTVYRCHTTGPSRAYMVDMEGARGGDAVTIATVCHTDTSLWNPEHVSFKLLGTKPGGTPVCHLMPYGHIIWAKNVNRSPA

SEQ ID NO: 41 - protein, 362 aa, Triticum aestivum
MARFLVALLAATLVAVQAGGQLGHAAPATGEVFWRAVLPHSPLPDAVLRLLKQPAAESTSFVRDPEDRPPFDYRDYSRSSSDDEPSKSTVAASGAGGFDYDNYSGADERRGATDEYKAPSSSLAGSGAYMARGGKAETTTVFFHEEAVRVGRRLPFHFPPATPAALGFLPRQVADSVPFTTAALPGILATFGIASDSTTVPSMEATLRACESPTIAGESKFCATSLEALVERAMGVLGTRDIRPVTSTLPRAGAPLQTYTVVAVQPVEGGPVFVACHDEAYPYTVYRCHTTGPSRAYTVDMEGARGADAVTIAAVCHTDTSLWNPEHVSFKLLGTKPGGTPVCHLMPYGHIIWAKNVKRSPA

SEQ ID NO: 42 - protein, 82 aa, Triticum aestivum
MARFLVALLAATLVAVQAGGQLGHAAPATAEVFWRAVLPHSPLPDAVLRLLKQPAAGVELHTEATSFVRDPEDRPPFDYRDY

SEQ ID NO: 43 - protein, 412 aa, Oryza sativa
MARFLLLLVAVAAAAAVLSLGDAAPSTAEVFWRAVLPESPLPDAFLRLLRPDTSFVVGKAEAAGGAARTGFPFDYTDYRGSDSPTTASGLDLAGDFGEPAPFGYDYSAQGEGGGGGAAAAAGEQVLAVDAGFNYDKYVGARKLRGGSSTAGGENDDEPFGYDYKAPSSGSGTAASTTARGVGTGATTTVFFHEEAVRVGERLPFYFPAATTSALGFLPRRVADSIPFTAAALPAVLALFGVAPDTAEAAGMRETLRTCEWPTLAGESKFCATSLEALVEGAMAALGTRDIAALASTLPRGGAPLQAYAVRAVLPVEGAGFVACHDQAYPYTVYRCHTTGPARAYMVEMEGDGGGDGGEAVTVATVCHTNTSRWNPEHVSFKLLGTKPGGSPVCHLMPYGHIVWAKNVKSSTA